(19) United States
(12) Patent Application Publication
(10) Pub. No.: US 2024/0331819 A1
(43) Pub. Date: Oct. 3, 2024

(54) MEDICAL INFORMATION PROCESSING APPARATUS, MEDICAL INFORMATION DISPLAY SYSTEM, AND MEDICAL INFORMATION PROCESSING METHOD

(71) Applicant: CANON MEDICAL SYSTEMS CORPORATION, Otawara-shi (JP)

(72) Inventors: Tomoko TAKAYAMA, Chuo (JP); Atsuko SUGIYAMA, Nasushiobara (JP); Katsuhiko FUJIMOTO, Saitama (JP); Mariko SHIBATA, Nasushiobara (JP); Hayato OKUMIYA, Nasushiobara (JP)

(73) Assignee: CANON MEDICAL SYSTEMS CORPORATION, Otawara-shi (JP)

(21) Appl. No.: 18/624,209

(22) Filed: Apr. 2, 2024

(30) Foreign Application Priority Data
Apr. 3, 2023 (JP) 2023-060392

(51) Int. Cl. G16H 10/60

(52) U.S. Cl. CPC G16H 10/60

(57) ABSTRACT

A medical information processing apparatus according to an embodiment includes processing circuitry. The processing circuitry is configured: to receive an input of a topic related to a targeted patient subject to a consultation/treatment process; to specify an item of a medical event relevant to the input topic and a reference period of the item, from time-series information recording medical events implemented for the targeted patient and time spans in each of which a state of a different one of items was observed from the targeted patient during the medical event, the items belonging to the medical events and being classified from a medical biological viewpoint and a viewpoint other than the medical biological viewpoint; and to output a specified result.

### CROSS-REFERENCE TO RELATED APPLICATIONS

This application is based upon and claims the benefit of priority from Japanese Patent Application No. 2023-060392, filed on Apr. 3, 2023, the entire contents of which are incorporated herein by reference.

### FIELD

Embodiments described herein relate generally to a medical information processing apparatus, a medical information display system, and a medical information processing method.

### BACKGROUND

Conventionally, medical providers such as medical doctors are required to perform consultation/treatment processes on the basis of not only biological aspects such as examination results, but also social aspects and psychological aspects of patients. For this reason, it has been practice to record, in electronic charts or the like, not only information about the biological aspects, but also information about the social aspects and the psychological aspects of patients, as consultation/treatment information.

### BRIEF DESCRIPTION OF THE DRAWINGS

FIG. 1 is a block diagram illustrating an exemplary configuration of a clinical assistance system according to an embodiment;

FIG. 2 is a block diagram illustrating an exemplary configuration of a clinical assistance apparatus according to the embodiment;

FIG. 3 is a drawing illustrating an exemplary data configuration of specification information according to the embodiment;

FIG. 4 is a drawing illustrating an exemplary screen configuration of a display screen according to the embodiment;

FIG. 5 is a drawing illustrating an exemplary screen configuration of another display screen according to the embodiment;

FIG. 6 is a drawing illustrating an exemplary screen configuration of yet another display screen according to the embodiment;

FIG. 7 is a drawing illustrating an exemplary screen configuration of yet another display screen according to the embodiment;

FIG. 8 is a drawing illustrating an exemplary screen configuration of yet another display screen according to the embodiment;

FIG. 9 is a chart illustrating an example of a process performed by the clinical assistance apparatus according to the embodiment;

FIG. 10 is a drawing illustrating an exemplary data configuration of specification information according to a first modification example;

FIG. 11 is a drawing illustrating an exemplary screen configuration of a display screen according to the first modification example;

FIG. 12 is a drawing illustrating an example of a graph structure of specification information according to a second modification example;

FIG. 13 is a block diagram illustrating an exemplary configuration of a clinical assistance apparatus according to a third modification example;

FIG. 14 is a drawing illustrating an exemplary screen configuration of a display screen according to the third modification example;

FIG. 15 is a block diagram illustrating an exemplary configuration of a clinical assistance apparatus according to a fourth modification example; and

FIG. 16 is a drawing illustrating an exemplary screen configuration of a display screen according to the fourth modification example.

### DETAILED DESCRIPTION

A medical information processing apparatus according to an embodiment includes processing circuitry. The processing circuitry is configured: to receive an input of a topic related to a targeted patient subject to a consultation/treatment process; to specify an item of a medical event relevant to the input topic and a reference period of the item, from time-series information recording medical events implemented for the targeted patient and time spans in each of which a state of a different one of items was observed from the targeted patient during the medical event, the items belonging to the medical events and being classified from a medical biological viewpoint and a viewpoint other than the medical biological viewpoint; and to output a specified result.

Exemplary embodiments of a medical information processing apparatus, a medical information display system, and a medical information processing method of the present disclosure will be explained below, with reference to the accompanying drawings. In the following embodiments, some of the elements that are referred to by using the same reference characters are assumed to perform the same or similar operations, and duplicate explanations thereof will be omitted as appropriate.

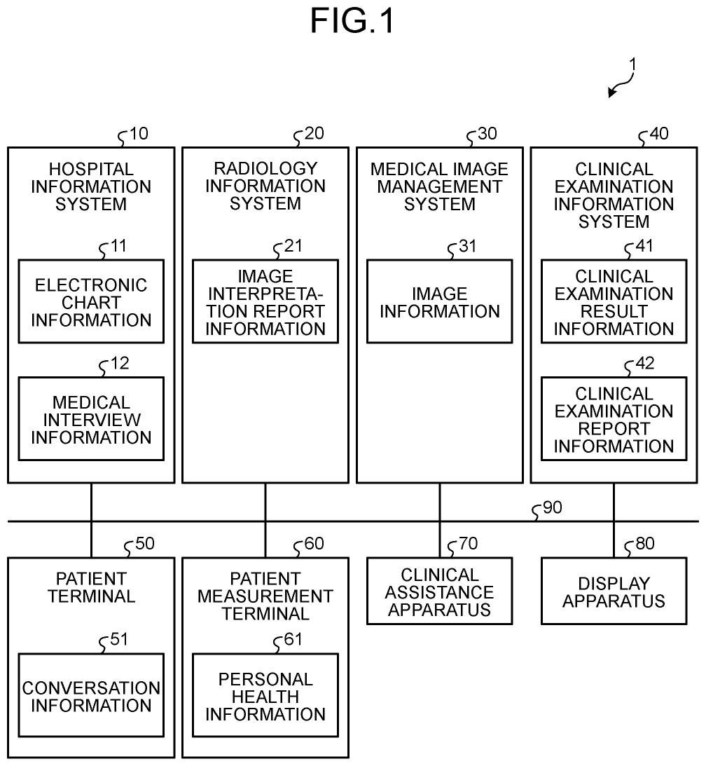

FIG. 1 is a block diagram illustrating an exemplary configuration of a clinical assistance system 1 according to an embodiment of the present disclosure. The clinical assistance system 1 is an example of a medical information processing system. The clinical assistance system 1 includes a Hospital Information System (HIS) 10, a Radiology Information System (RIS) 20, a medical image management system (called a Picture Archiving and Communication System (PACS)) 30, a clinical examination information system (called a Laboratory Information System (LIS)) 40, a patient terminal 50, a patient measurement terminal 60, a clinical assistance apparatus 70, and a display apparatus 80.

Further, the systems and the apparatuses are connected so as to be able to communicate with one another via a network 90. The configuration illustrated in FIG. 1 is merely an example, and the quantities of the systems and the apparatuses may arbitrarily be changed. Further, other apparatuses that are not illustrated in FIG. 1 may be connected to the network 90.

The hospital information system 10, the radiology information system 20, the medical image management system 30, and the clinical examination information system 40 are each realized by using a computer machine such as a server or a workstation, for example.

The hospital information system 10 is configured to store therein electronic chart information 11 and medical interview information 12. The electronic chart information 11 is information recording therein consultation/treatment progress of a patient. For example, the electronic chart information 11 includes information for identifying the patient and personal information such as the age, the gender, and a family structure of the patient, as well as information about the names of diseases the patient has suffered from, the names of prescribed drugs, a treatment time period, and the like. The medical interview information 12 is information indicating details of the patient's responses in medical interviews. For example, the medical interview information 12 includes the patient's responses about current symptoms, the patient's responses about his/her living environment, and the patient's responses in a personality analysis.

The radiology information system 20 is configured to store therein image interpretation report information 21. The image interpretation report information 21 is information including observations of a medical doctor or the like who interpreted image information 31 taken by a medical image diagnosis apparatus.

The medical image management system 30 is configured to store therein the image information 31. The image information 31 includes one or more images taken by the medical image diagnosis apparatus. The medical image diagnosis apparatus is an apparatus such as an X-ray Computed Tomography (CT) apparatus, a Magnetic Resonance Imaging (MRI) apparatus, an X-ray diagnosis apparatus, or an ultrasound diagnosis apparatus, for example. Further, the image information 31 is compliant with a Digital Imaging and Communications in Medicine (DICOM) standard.

The clinical examination information system 40 is configured to stored therein clinical examination result information 41 and clinical examination report information 42. The clinical examination result information 41 is information indicating a result of a clinical examination. The clinical examination report information 42 is information indicating observations on the clinical examination result.

The patient terminal 50 is a terminal used by the patient. The patient terminal 50 is realized by using, for example, a smartphone, a tablet terminal, a wearable terminal, or a personal computer. Further, the patient terminal 50 is configured to store therein conversation information 51 recording conversations of the patient.

For example, the conversation information 51 is information about the conversations between one or more medical providers such as a medical doctor, a nurse, and/or a counselor and the patient. Further, the conversation information 51 may include not only conversations related to consultation/treatment details, but also small talk. More specifically, the conversation information 51 includes audio information recording the conversations and/or text information converted from audio. Further, the conversation information 51 may include picture information such as a video or a still image containing the conversations. When including the picture information, the conversation information 51 includes one or more pictures from which facial expressions of the patient can be observed.

The patient measurement terminal 60 is a terminal configured to measure vital signs of the patient. For example, the patient measurement terminal 60 is realized by using a wearable terminal. The patient measurement terminal 60 is configured to measure the vital signs such as blood pressure, a pulse rate, a body temperature, respiration, and/or the like. Further, the patient measurement terminal 60 may also be configured to record conversations. Further, the patient measurement terminal 60 is configured to store therein personal health information 61 including measurement results of the vital signs.

In this situation, the patient measurement terminal 60 does not necessarily need to be a wearable terminal and may be a body fat scale, an activity tracker, or other apparatuses. Also, the patient measurement terminal 60 may be configured to measure body fat, to measure an activity amount, and/or to measure other factors. Further, the clinical assistance system 1 may include two or more patient measurement terminals 60. Further, in the clinical assistance system 1, the two or more patient measurement terminals 60 may be configured to measure mutually-different factors.

The clinical assistance apparatus 70 is an apparatus configured to assist medical providers such as medical doctors, in clinical situations. The clinical assistance apparatus 70 is an example of the medical information processing apparatus. The clinical assistance apparatus 70 may be realized by using a computer machine such as a server or a workstation, for example.

The clinical assistance apparatus 70 is configured to assist a medical provider in efficiently browsing information by presenting the medical provider with a summary of various types of information generated on the basis of information obtained from any of the systems and the apparatuses in the clinical assistance system 1. The clinical assistance apparatus 70 may be provided in a facility or may be a server on the Internet. A configuration of the clinical assistance apparatus 70 will be explained later.

The display apparatus 80 is an apparatus capable of displaying various types of information. The display apparatus 80 is an example of the medical information display apparatus. The display apparatus 80 is realized by using a computer machine such as a personal computer or a tablet terminal. The display apparatus 80 is configured to display various types of information generated by the clinical assistance apparatus 70. Information displayed by the display apparatus 80 will be explained later.

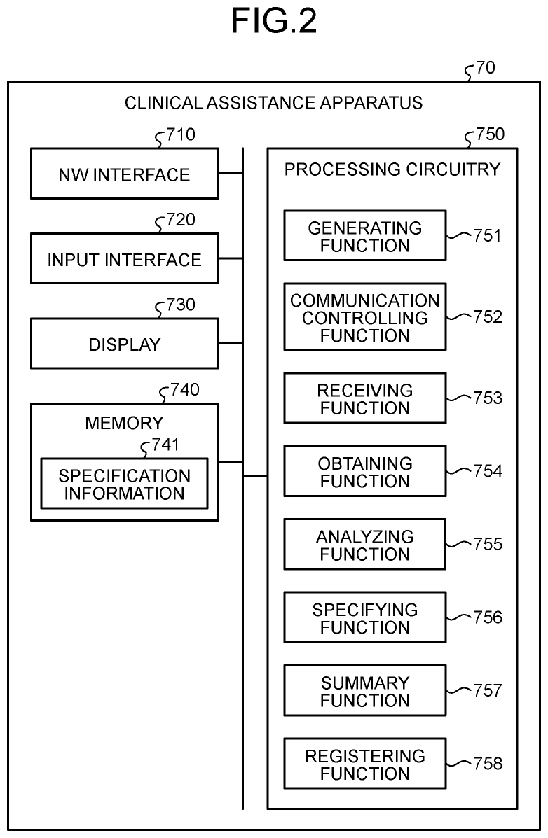

Next, a configuration of the clinical assistance apparatus 70 will be explained. FIG. 2 is a block diagram illustrating an exemplary configuration of the clinical assistance apparatus 70 according to the present embodiment. The clinical assistance apparatus 70 includes a network (NW) interface 710, an input interface 720, a display 730, a memory 740, and processing circuitry 750.

The NW interface 710 is connected to the processing circuitry 750 and is configured to control transfer of various types of data and communication performed with apparatuses connected via the network 90. For example, the NW interface 710 is realized by using a network card, a network adaptor, a Network Interface Controller (NIC), or the like.

The input interface 720 is connected to the processing circuitry 750 and is configured to convert input operations received from an operator (a medical provider) into electrical signals and to output the electrical signals to the processing circuitry 750. More specifically, the input interface 720 is configured to convert the input operations received from the operator into the electrical signals and to output the electrical signals to the processing circuitry 750.

For example, the input interface 720 is realized by using a trackball, a switch button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like.

In the present disclosure, the input interface 720 does not necessarily need to include physical operation component parts such as a mouse, a keyboard, and/or the like. For instance, possible examples of the input interface 720 include electrical signal processing circuitry configured to receive an electrical signal corresponding to an input operation from an external input mechanism provided separately from the apparatus and to output the electrical signal to controlling circuitry.

The display 730 is connected to the processing circuitry 750 and is configured to display various types of information and various types of image data output from the processing circuitry 750. For example, the display 730 is realized by using a liquid crystal display, a Cathode Ray Tube (CRT) display, an organic electroluminescence (EL) display, a plasma display, a touch panel, or the like.

The memory 740 is connected to the processing circuitry 750 and is configured to store therein various types of data. Also, the memory 740 is configured to store therein various types of programs for realizing various types of functions as being read and executed by the processing circuitry 750. For example, the memory 740 is realized by using a semiconductor memory element such as a Random Access Memory (RAN) or a flash memory, or a hard disk, an optical disk, or the like.

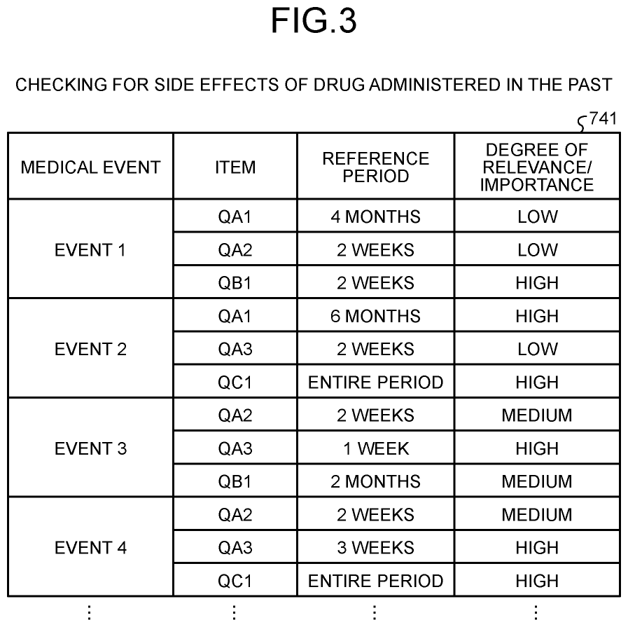

For example, the memory 740 is configured to store therein specification information 741. FIG. 3 is a drawing illustrating an exemplary data configuration of the specification information 741. The specification information 741 is information for specifying one or more items corresponding to a topic and small events related to the items. The specification information 741 is an example of correspondence information.

The specification information 741 is information in which, with respect to each of various reference purposes of the consultation/treatment information (which hereinafter may simply be referred to as “reference purposes”), medical events, items, reference periods, and degrees of relevance/importance are kept in correspondence with one another. The specification information 741 is determined, for example, on the basis of medical papers, academic books, clinical study records, treatment guidelines, accumulated knowledge, and/or the like.

Each of the reference purposes indicates a purpose for which the consultation/treatment information related to the targeted patient is to be referenced. Examples of the reference purposes include: checking for side effects of a drug administered in the past, considering administration of a new drug, examining a treatment plan for the future, checking consultation/treatment actions up to the last time, and making a proposal about the patient's lifestyle (eating, work, recreation).

FIG. 3 illustrates a correspondence relationship among medical events, items, reference periods, and degrees of relevance/importance with respect to a reference purpose of “checking for side effects of a drug administered in the past”. The example in FIG. 3 illustrates the correspondence relationship with respect to only the single reference purpose. However, it is assumed that a correspondence relationship among medical events, items, reference periods, and degrees of relevance/importance is defined with respect to each of various reference purpose.

The medical events represent medical actions taken for the patient in the past, medical-related events related to the patient, and the like. The medical events denote, for example, surgery, administration of a drug, performing radiation treatment, and the like. The reference purposes and the medical events are examples of the topic.

The items represent, among pieces of consultation/treatment information managed in the clinical assistance system 1, pieces of consultation/treatment information relevant to the medical events. When a plurality of items are kept in correspondence with the medical events, there are correlational relationships and cause-effect relationships among the plurality of items. Examples of the items include, for instance, items classified from a medical biological viewpoint, items classified from a psychological viewpoint, and items classified from a social viewpoint.

More specifically, examples of the items classified from the medical biological viewpoint include information related to medical symptoms occurring in the patient's body such as a pain in the body, nausea, vomiting, numbness in the body, fatigue, tiredness, and/or the like. Further, examples of the items classified from the psychological viewpoint include information related to a mental status of the patient such as feeling depressed, interference with daily life, and/or the like. Further, examples of the items classified from the social viewpoint include information related to the patient's life such as his/her family structure, work, financial situation, hobbies, and/or the like.

The reference period denotes an optimal reference period for small events related to each of the items relevant to the medical events corresponding to the reference purpose. The small events denote events that occur along with implementation of a medical event. In other words, by implementing a plurality of small events, the implementation of the medical event is achieved.

In an example, the reference period may be defined by taking into account a blank period in which no consultation/treatment was performed for a targeted patient subject to consultation/treatment processes. For example, the reference periods may be defined so as to be longer when there has been a two-year blank period than when there has been a one-year blank period.

The small events are records of time spans in each of which the state of a different one of the items was observed from the targeted patient being subject to a consultation/treatment process. Among the small events, a certain small event related to an item (an example of a first item) classified from the medical biological viewpoint serves as an example of a first event, whereas another small event related to an item (an example of a second item) classified from another viewpoint other than the medical biological viewpoint such as the psychological viewpoint or the social viewpoint serves as an example of a second event.

The degrees of relevance/importance indicate degrees of importance of the items with respect to the medical events and degrees of relevance between the medical events and the items, in accordance with the reference purposes. The degrees of relevance/importance are examples of the weight. Although a degree of relevance and a degree of importance together serve as one index in the example in FIG. 3, the specification information 741 may have a degree of importance and a degree of relevance as separate indices that are kept in correspondence with other information. Further, the abovementioned reference periods may be defined while taking the degrees of relevance/importance into account.

In FIG. 3, the first line indicates that “event 1” is relevant to the item named “item QA1”. Further, in FIG. 3, the first line indicates that the reference period of the medical event related to the “item QA1” is “from the present to two months ago”, while the degree of relevance/importance of the “item QA1” is “medium”.

In this situation, FIG. 3 illustrates an example in which the reference period denotes, with respect to each of the items, how much earlier than the present time (up to which point in time) the small events should be referenced; however, possible methods for defining the reference periods are not limited to this example. For instance, when side effects of a drug administered in the past are checked for, the reference period may indicate how much earlier than termination of the administration of the drug (up to which point in time), the small events should be referenced.

Further, although FIG. 3 indicates the degrees of relevance/importance as high, medium, and low, possible methods for defining the degrees of relevance/importance are not limited to this example. For instance, the degrees of relevance/importance may be expressed with five grades using the numerals 1 to 5.

As an example, a situation will be explained in which a supervisor of the clinical assistance system 1 sets, as the specification information 741, items classified from the medical biological viewpoint and reference periods thereof, with respect to a medical event of “administering drug A” relevant to a reference purpose of “checking for side effects”. In this situation, the supervisor specifies items relevant to the side effects that may occur due to the administration of drug A, from what is written in a paper or the like describing the side effects of drug A and further sets the items as “items” in the specification information 741. In addition, with regard to the side effects that may occur due to the administration of drug A, the supervisor specifies how long after the drug administration the side effects may occur, on the basis of what is written in a paper or the like and further sets the time period as a “reference period”.

Further, when setting degrees of relevance/importance in the above example, the supervisor specifies, with respect to the side effects that may occur due to the administration of drug A, how often the side effects may occur on the basis of what is written in a paper or the like and further sets the degrees of relevance/importance in such a manner that the more often a side effect occurs, the higher is the degree of relevance/importance thereof. Further, in this situation, the supervisor may set a higher degree of relevance/importance for a side effect that occurs less frequently, but may be more serious in the event of occurrence.

Next, with the above example, a situation will be explained in which an item classified from the psychological viewpoint and a reference period thereof are set as the specification information 741. In this situation, on the basis of mental tendencies (having increasing anxiety, being unable to suppress irritation, easily feeling sad, etc.) that may appear in patients for whom drug A was administered, the supervisor specifies the “item” classified from the psychological viewpoint relevant to the administration of drug A. Further, the supervisor sets the “reference period” on the basis of how long after the drug administration the abovementioned mental tendencies are likely to start appearing, or the like.

Next, with the above example, another situation will be explained in which an item classified from the social viewpoint and a reference period thereof are set as the specification information 741. In this situation, on the basis of a daily activity or the like (e.g., becoming unable to drive a car due to drowsiness caused thereby) expected to be hindered by the administration of drug A, the supervisor specifies the “item” classified from the social viewpoint relevant to the administration of drug A. Further, the supervisor sets the “reference period” on the basis of a time period considered to be sufficient for understanding information about the abovementioned daily activity.

With reference to the specification information 741, a specifying function 756 (explained later) of the processing circuitry 750 is capable, on the basis of a reference purpose input by the medical provider, of specifying medical events relevant to the reference purpose, items, and reference periods related to the items.

Returning to the description of FIG. 2, the processing circuitry 750 is configured to control operations of the entirety of the clinical assistance apparatus 70. As functional units thereof, the processing circuitry 750 includes, for example, a generating function 751, a communication controlling function 752, a receiving function 753, an obtaining function 754, an analyzing function 755, the specifying function 756, a summary function 757, and a registering function 758.

In an embodiment, processing functions executed by the constituent elements, namely, the generating function 751, the communication controlling function 752, the receiving function 753, the obtaining function 754, the analyzing function 755, the specifying function 756, the summary function 757, and the registering function 758, are stored in the memory 740 in the form of computer-executable programs.

The processing circuitry 750 is a processor configured to realize the functions corresponding to the programs, by reading and executing the programs from the memory 740. In other words, the processing circuitry 750 that has read the programs has the functions illustrated within the processing circuitry 750 in FIG. 2.

Although FIG. 2 illustrates the example in which the single processor is configured to realize the generating function 751, the communication controlling function 752, the receiving function 753, the obtaining function 754, the analyzing function 755, the specifying function 756, the summary function 757, and the registering function 758, it is also acceptable to structure the processing circuitry 750 by combining together a plurality of independent processors, so that the functions are realized as a result of the processors executing the programs.

Further, although FIG. 2 illustrates the example in which the single memory such as the memory 740 is configured to store therein the programs corresponding to the processing functions, it is also acceptable to provide a plurality of memories in a distributed manner, so that the processing circuitry 750 is configured to read corresponding programs from the individual memories.

The term “processor” used in the above explanations denotes, for example, a Central Processing Unit (CPU), a Graphical Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)).

The one or more processors are configured to realize the functions by reading and executing the programs saved in the memory 740. Alternatively, instead of having the programs saved in the memory 740, it is also acceptable to directly incorporate the programs into the circuitry of the one or more processors. In that situation, the one or more processors are configured to realize the functions by reading and executing the programs incorporated in the circuitry thereof.

The generating function 751 is configured to generate various types of display screens to be displayed on the display apparatus 80. The various types of display screens generated by the generating function 751 will be explained later.

The communication controlling function 752 is configured to control communication between the clinical assistance apparatus 70 and other apparatuses. For example, the communication controlling function 752 is configured to transmit and receive various types of information to and from the other apparatuses in the clinical assistance system 1, via the NW interface 710. As an example, the communication controlling function 752 is configured to transmit any of the display screens generated by the generating function 751 to the display apparatus 80.

The receiving function 753 is configured to receive inputs of various types of information. For example, the receiving function 753 is configured to receive an input designating the targeted patient, from the medical provider via the display apparatus 80 and the communication controlling function 752. As an example, the receiving function 753 is configured to receive an input of information that makes it possible to identify the patient, such as the full name of the patient or a patient code identifying the patient.

In this situation, the receiving function 753 may be configured to receive the input designating the targeted patient that is selected from a list of patients. Further, the receiving function 753 may be configured to receive an input of various types of information directly from a user via the input interface 720. In that situation, the various types of information may be displayed on the display 730 of the clinical assistance apparatus 70.

Further, for example, the receiving function 753 is configured to receive an input of the reference purpose related to the targeted patient, from the medical provider via the display apparatus 80. In that situation, the reference purpose received by the receiving function 753 is used for a process of browsing summaries.

Further, for example, the receiving function 753 is configured to receive inputs of reference purposes, medical events, items, reference periods, and degrees of relevance/importance, from the medical provider via the display apparatus 80. In that situation, the reference purposes and the like received by the receiving function 753 are used in processes of registering and editing the specification information.

The obtaining function 754 is configured to obtain the consultation/treatment information related to the targeted patient. For example, by controlling the communication controlling function 752, the obtaining function 754 is configured to obtain, as the consultation/treatment information, patient information related to the patient, the medical interview information 12 indicating the details of the patient's responses in medical interviews, and the conversation information 51 recording conversations of the patient, from any of the apparatuses structuring the clinical assistance system 1.

In this situation, the patient information is information obtained by monitoring the targeted patient, such as the electronic chart information 11, the image interpretation report information 21, the image information 31, the clinical examination result information 41, clinical examination report information 42, the personal health information 61, and/or the like. For example, when the receiving function 753 has received an input designating the targeted patient, the obtaining function 754 is configured to obtain the patient information, the medical interview information 12, and the conversation information 51 of the designated targeted patient, as the consultation/treatment information.

The analyzing function 755 is configured to analyze the consultation/treatment information obtained by the obtaining function 754. For example, the analyzing function 755 is configured to divide pieces of consultation/treatment information obtained by the obtaining function 754 into large categories such as “medical adaptations”, “patient's intentions”, “Quality Of Life (QOL)” and “peripheral matters”. The pieces of consultation/treatment information correspond to the “items” in the specification information 741.

In this situation, for example, the “medical adaptations” denote medical information such as the size of cancer, whether metastasis is present or absent, what type of cancer treatment is being performed, and/or the like. The “patient's intentions” denote information related to intentions of the examined subject such as, in an example of breast cancer, whether the patient places importance on breast conservation or places importance on treatment effects. The “QOL” denotes information related to quality of lifestyle or life of the examined subject. The “peripheral matters” denote information related to the surroundings of the examined subject such as his/her family structure. In this situation, each of the pieces of consultation/treatment information may be divided into a plurality of large categories.

In the present embodiment, items classified from the medical biological viewpoint belong to the “medical adaptations”; items classified from the psychological viewpoint and the social viewpoint belong to the “patient's intentions”; items classified from the medical biological viewpoint, the psychological viewpoint, and the social viewpoint belong to the “QOL”, and items classified from the social viewpoint belong to the “peripheral matters”.

As a method for dividing the consultation/treatment information, for example, a method may be used by which each of the pieces of consultation/treatment information is set with one of tags respectively representing “medical adaptations”, “patient's intentions” “QOL”, and “peripheral matters”, so that each of the pieces of consultation/treatment information is classified into one of the large categories such as “medical adaptations”, “patient's intentions” “QOL”, and “peripheral matters”, according to the tags. In that situation, as for the tag setting process, the tags may be automatically set by using a publicly-known natural language processing technique or the like or may be manually set by the user.

Alternatively, it is also acceptable to store, in the memory 740 or the like, correspondence information keeping item names in correspondence with large category names, so that the pieces of consultation/treatment information are divided on the basis of item names indicating the names of the pieces of consultation/treatment information and the stored information.

The consultation/treatment information divided by the analyzing function 755 is managed by using a format in which, with respect to each of the large categories, small events are arranged in a chronological order regarding each of the items, the small events each recording a time span in which the state corresponding to the item was observed from the targeted patient. The consultation/treatment information in this situation is an example of the time-series information.

Further, for example, the analyzing function 755 is configured to further divide the items that have been divided into the large categories, on the basis of the medical biological viewpoint, the psychological viewpoint, and the social viewpoint (which hereinafter may be referred to as “small categories”). In an example, the analyzing function 755 may be configured to divide the pieces of consultation/treatment information on the basis of the medical biological viewpoint and the other viewpoints. For the small categories also, it is possible to use the same methods as those used for the division into the large categories.

Further, the analyzing function 755 is configured to calculate an importance score with respect to each of the items. The importance score quantitatively expresses importance of each of the items for the targeted patient. For example, the analyzing function 755 is configured to calculate the importance score on the basis of the state of each of the items for the targeted patient, according to an evaluation index determined in advance for each item. As a result of calculating the importance scores, it becomes possible to directly compare the importance among the items.

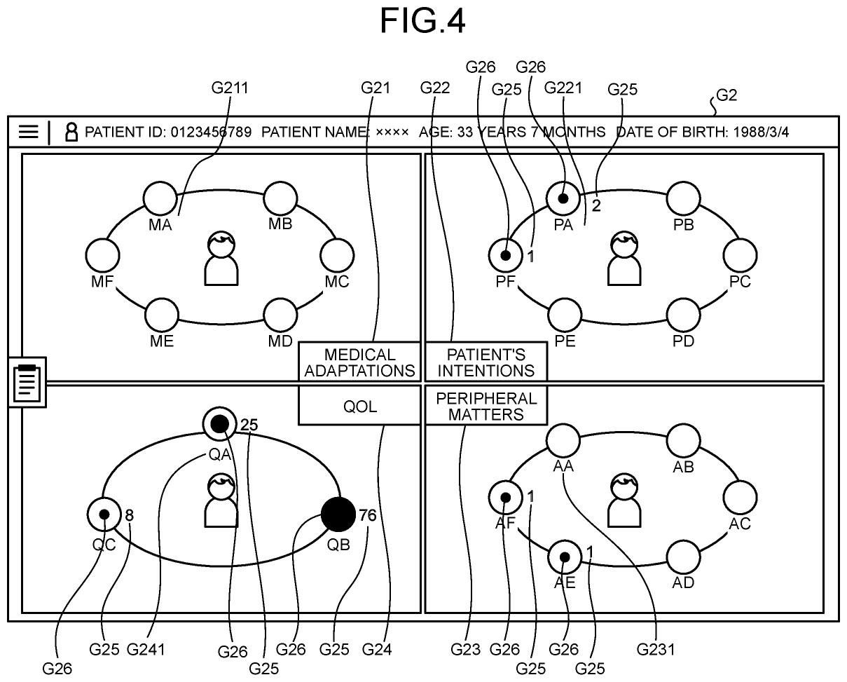

In relation to the above, FIG. 4 is a drawing illustrating an exemplary screen configuration of a first display screen G2. The first display screen G2 is an example of a screen generated by the generating function 751 and displays a small category importance score with respect to each of the items divided into the large categories such as the medical adaptations, the patient's intentions, the peripheral matters, and the QOL.

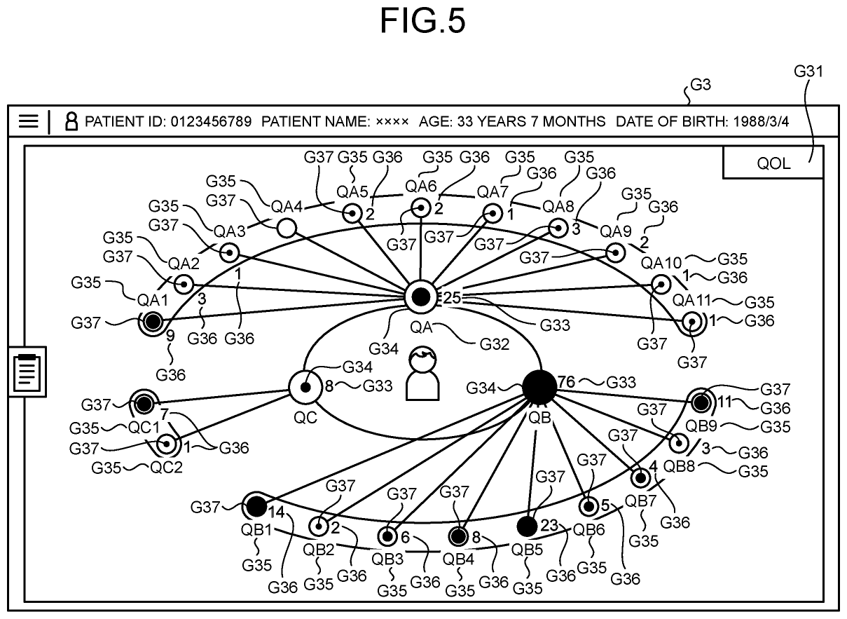

A first display region G211 is a region for displaying small category names corresponding to the medical adaptations. The example in FIG. 4 indicates that the medical adaptations are further divided into the small categories named MA, MB, MC, MD, ME, and MF. A first button G21 is a button used for receiving, from the medical provider, an operation to cause a second display screen G3 (see FIG. 5) corresponding to the first display region G211 to be displayed.

A second display region G221 is a region for displaying small category names corresponding to the patient's intentions. The example in FIG. 4 indicates that the patient's intentions are further divided into the small categories named PA, PB, PC, PD, PE, and PF. A second button G22 is a button used for receiving, from the medical provider, an operation to cause a second display screen G3 (see FIG. 5) corresponding to the second display region G221 to be displayed.

A third display region G231 is a region for displaying small category names corresponding to the peripheral matters. The example in FIG. 4 indicates that the peripheral matters are further divided into the small categories named AA, AB, AC, AD, AE, and AF. A third button G23 is a button used for receiving, from the medical provider, an operation to cause a second display screen G3 (see FIG. 5) corresponding to the third display region G231 to be displayed.

A fourth display region G241 is a region for displaying small category names corresponding to the QOL. The example in FIG. 4 indicates that the QOL is further divided into the small categories named QA, QB, and QC. A fourth button G24 is a button used for receiving, from the medical provider, an operation to cause a second display screen G3 (see FIG. 5) corresponding to the fourth display region G241 to be displayed.

The scores G25 indicate the importance scores in units of the small categories. For example, each of the scores G25 may be a numerical value of the sum of the importance scores of the items divided into the small categories. Alternatively, each of the scores G25 may be a maximum value, an average value, or the like of the importance scores of the items divided into the small categories.

The example in FIG. 4 indicates that, in the second display region G221, the importance score of the small category PA is 2; the importance score of the small category PF is 1; for the peripheral matters, the importance scores of the small category AE and the small category AF are each 1; for the QOL, the importance score of the small category QA is 25, the importance score of the small category QB is 76, and the importance score of the small category QC is 8. As for the items having no numerical values, it means that there is no information related to those items.

Guides G26 are displayed elements indicating degrees of importance of the small categories. In the example in FIG. 4, the guides G26 are displayed while the sizes of the black dots in the circular regions are varied in accordance with the numerical values of the scores G25. With this arrangement, it is easier for the medical provider to understand the importance of each of the items.

In the example in FIG. 4, the display regions representing the large categories indicate the small category names and the scores; however, what can be displayed in the display regions representing the large categories is not limited to those examples. For instance, a part of the displayed content described above does not necessarily need to be included, and some other displayed content may be included.

When a button is selected from among the first button G21, the second button G22, the third button G23, and the fourth button G24, the generating function 751 is configured to generate the second display screen G3 corresponding to the selected button.

In relation to the above, FIG. 5 is a drawing illustrating an exemplary screen configuration of the second display screen G3. second display screen G3 is a screen generated by the generating function 751 for displaying importance scores in units of the small categories and an importance score of each of the items.

The second display screen G3 includes, as a screen configuration thereof, a large category display region G31, a small category display region G32, small category scores G33, first guides G34, item display regions G35, item scores G36, and second guides G37.

The large category display region G31 is a region for displaying a large category name. In the example in FIG. 5, the large category display region G31 indicates that the second display screen G3 corresponds to the QOL.

The small category display region G32 is a region for displaying the small category names in the large category displayed in the large category display region G31. Similarly to the first display screen G2 in FIG. 4, the example in FIG. 5 indicates that the QOL is further divided into the small categories named QA, QB, and QC.

The small category scores G33 indicate the importance scores in units of the small categories. Similarly to the first display screen G2 in FIG. 4, the example in FIG. 5 indicates that, in the QOL, the importance score of the small category QA is 25; the importance score of the small category QB is 76; and the importance score of the small category QC is 8.

The first guides G34 are displayed elements indicating degrees of importance of the small categories. Similarly to the first display screen G2 in FIG. 4, in the example in FIG. 5, the first guides G34 are displayed while the sizes of the black dots in the circular regions are varied in accordance with the numerical values of the small category scores G33.

The item display regions G35 are regions for displaying the item names in the small categories connected by leader lines. The example in FIG. 5 indicates that the items divided into a small category called the small category QA are items QA1 to QA11; the items divided into a small category called the small category QB are items QB1 to QB9; and the items divided into a small category called the small category QC are items QC1 to QC3.

Further, in the example in FIG. 5, the small category names in the small category display regions G32 and the item names in the item display regions G35 are displayed while being connected by leader lines. As a result, the medical provider is able to easily understand relationships between the small categories and the items.

The item scores G36 indicate the importance scores of the items. The example in FIG. 5 indicates that, as for the importance scores of the items in the small category QA, 9 for the item QA1, 3 for the item QA2, 1 for the item QA3, 2 for the item QA5, 2 for the item QA6, 1 for the item QA7, 3 for the item QA8, 2 for the item QA9, 1 for the item QA10, and 1 for the item QA11. Further, 25 being the value of the small category score G33 of the small category QA denotes the sum of the item scores G36 of the items divided into the small category QA.

Further, as for the importance scores of the items in the small category QB, 14 for the item QB1, 2 for the item QB2, 6 for the item QB3, 8 for the item QB4, 23 for the item QB5, 5 for the item QB6, 4 for the item QB7, 3 for the item QB8, and 11 for the item QB9. Further, 76 being the value of the small category score G33 of the small category QB denotes the sum of the item scores G36 of the items divided into the small category QB.

Also, as for the importance scores of the items in the small category QC, 7 for the item QC1 and 1 for the item QC2. Also, 8 being the value of the small category score G33 of the small category QC denotes the sum of the item scores G36 of the items divided into the small category QC.

The second guides G37 are displayed elements indicating degrees of importance of the items. In the example in FIG. 5, the second guides G37 are displayed while the sizes of the black dots in the circular regions are varied in accordance with the numerical values of the item scores G36.

The explanation will be continued, returning to the description of FIG. 2. The specifying function 756 is configured to specify certain items of the medical events that were implemented for the targeted patient and are relevant to the input reference purpose, as well as the reference periods of the items, from the consultation/treatment information obtained by the obtaining function 754.

For example, the receiving function 753 is configured to receive, from the medical provider, an input of the reference purpose of the consultation/treatment information related to the targeted patient, via a reference purpose input screen (not illustrated). In an example, when the medical provider has input an instruction to display the reference purpose input screen via an operation menu (not illustrated) or the like displayed on the display apparatus 80, the reference purpose input screen is displayed on the display apparatus 80 by the generating function 751 and the communication controlling function 752.

Subsequently, the specifying function 756 is configured to specify the medical events, the items, the reference periods, and the degrees of relevance/importance corresponding to the received reference purpose, by referring to the specification information 741 in the memory 740. In an example, a situation will be explained in which an input of “checking for side effects of a drug administered in the past” is received as the reference purpose, so that the medical events, the items, the reference periods, and the degrees of relevance/importance are specified by using the specification information 741 illustrated in FIG. 3.

In this situation, by referring to the specification information 741 in FIG. 3, the specifying function 756 is configured to specify “event 1”, “event 2”, “event 3”, “event 4” and so on corresponding to “checking for side effects of a drug administered in the past”, as the medical events relevant to “checking for side effects of a drug administered in the past”. Subsequently, the specifying function 756 is configured to specify the medical events implemented for the targeted patient, by referring to the consultation/treatment information obtained by the obtaining function 754.

After that, the specifying function 756 is configured to compare the medical events relevant to “checking for side effects of a drug administered in the past” with the medical events implemented for the targeted patient. Subsequently, from among the medical events relevant to “checking for side effects of a drug administered in the past”, the specifying function 756 is configured to specify the medical events implemented for the targeted patient.

In this situation, when two or more medical events that have already been implemented are specified, the specifying function 756 may be configured to receive a designation of one of the medical events from the medical provider or may be configured to specify one of the medical events according to a predetermined condition, such as a medical event that occurred most recently prior to the present time. Further, regarding the specified plurality of medical events, the specifying function 756 may be configured to present the medical provider with specified results that are divided in correspondence with the medical events.

In an example, when “event 1” has already been implemented for the targeted patient, the specifying function 756 specifies “event 1” as a medical event that has already been implemented for the targeted patient and is relevant to “checking for side effects of a drug administered in the past”. Further, by referring to the specification information 741, the specifying function 756 specifies the “item QA1”, the “item QA2”, and the item “QB1” corresponding “event 1” that was specified, as the items relevant to “event 1”.

Also, similarly, the specifying function 756 specifies the reference period of the event related to the “item QA1” as “two months”, the reference period of the event related to the “item QA2” as “two weeks”, and the reference period of the event related to the “item QB1” as “two weeks”.

Further, similarly, the specifying function 756 specifies the degree of relevance/importance of the “item QA1” with “event 1” as “medium”, the degree of relevance/importance of the “item QA2” with “event 1” as “low”, and the degree of relevance/importance of the “item QB1” with “event 1” as “low”.

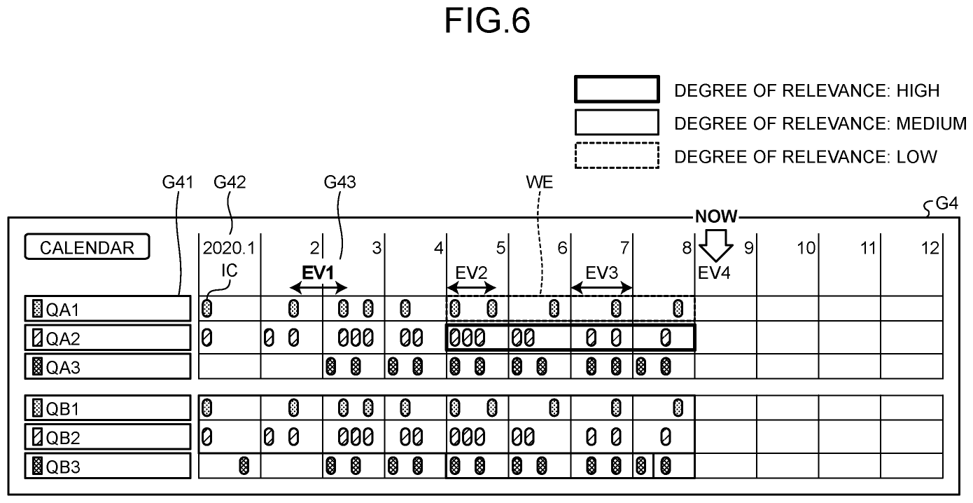

In relation to the above, FIG. 6 illustrates an exemplary screen configuration of a specified result display screen G4. The specified result display screen G4 is generated by the generating function 751 in accordance with the specified results obtained by the specifying function 756. The specified result display screen G4 may be displayed alone or may be displayed together with one or more other screens such as the second display screen G3.

In the example in FIG. 6, the specified result display screen G4 includes, as a screen configuration thereof, an item display region G41, a calendar display region G42, an event display region G43, event icons IC, and a display frame WE.

The item display region G41 is a region for displaying the item names of the items specified as the items relevant to the medical events and the reference purpose. In the example in FIG. 6, six item names such as “item QA1”, “item QA2”, “item QA3”, “item QB1”, “item QB2”, and “item QB3” are displayed.

The calendar display region G42 is a region for displaying occurrence time spans of the small events. In the example in FIG. 6, the calendar display region G42 displays a calendar of January to December, 2020. Further, the text “NOW” in the calendar display region G42 is a displayed element indicating the present time.

The event display region G43 is a region for displaying event names of the medical events. Further, the display positions of the event names indicate the time periods in which the medical events occurred. The example in FIG. 6 indicates that “event (EV) 1” occurred in the time period from mid-February to mid-March in 2020. Further, displaying “event 1” with an emphasis indicates that “event 1” is a medical event specified on the basis of the reference purpose input by the medical provider.

Further, in the example in FIG. 6, “event 4” is displayed underneath the text “NOW”. It is indicated that “event 4” is scheduled to start being implemented in the future some time from August to September in 2020.

The event icons IC are icons representing the small events that occurred. The example in FIG. 6 indicates that, in January 2020, a small event related to the “item QA1” occurred once; a small event related to the “item QA2” occurred once; a small event related to the “item QB1” occurred once; a small event related to the “item QB2” occurred once; and a small event related to the “item QB3” occurred once.

Further, the display positions of the event icons IC indicate the points in time at which the small events corresponding to the event icons IC occurred. The example in FIG. 6 indicates that the small events related to the “item QA1”, the “item QA2”, the “item QB1”, and the “item QB2” occurred in early January 2020 and the small event related to the “item QB3” occurred in late January. With this configuration, it is possible to express the occurrences of the small events chronologically.

In an example, the specified result display screen G4 may be configured so that, when an input to select one of the event icons IC is received (e.g., one of the event icons IC is clicked with an input mechanism such as a mouse) from the medical provider, details of the small event (e.g., the occurrence date/time of the small event in detail, the description of the small event, etc.) are displayed.

The display frame WE is a frame for indicating the reference periods and the degrees of relevance/importance of the small events related to the item relevant to the medical event specified by the specifying function 756.

In the present example, a situation will be explained in which “checking for side effects of a drug administered in the past” is input as an example of the reference purpose. In this situation, FIG. 6 indicates that the reference period of the small events related to the “item QA1” relevant to “event 1” corresponding to “checking for side effects of a drug administered in the past” is “four months from May to August”. Further, FIG. 6 indicates that the degree of relevance/importance between “event 1” corresponding to “checking for side effects of a drug administered in the past” and the “item QA1” is “low”.

In this situation, instead of indicating the reference period and the degree of relevance/importance, the display frame WE may be displayed by using a mode that makes it possible to distinguish whether each of the items is an item classified from the medical biological viewpoint, an item classified from the psychological viewpoint, or an item classified from the social viewpoint.

The explanation will be continued, returning to the description of FIG. 2. The summary function 757 is configured to generate summaries related to the consultation/treatment information to be presented to the medical provider. In the present embodiment, each of the summaries is obtained by summarizing main points of a corresponding one of the pieces of consultation/treatment information.

For example, with respect to the items specified by the specifying function 756, the summary function 757 is configured to generate each of the summaries, on the basis of information about small events related to the item implemented (or having occurred) during the specified reference period of the small events.

Further, for example, the summary function 757 is configured to determine a display order of the summaries in accordance with the degrees of relevance/importance. In an example, the summary function 757 is configured to determine the display order so that the higher the degree of relevance/importance of an item is, the closer to the top of the screen the summary thereof is displayed. Further, when there is an item of which the degree of relevance/importance does not satisfy a predetermined condition (e.g., being medium or higher), the summary function 757 may be configured not to generate the summary of such an item. Further, in that situation, such an item does not necessarily need to be displayed on the specified result display screen G4.

To generate the summaries, it is possible to use a publicly-known method, as appropriate. The summary function 757 is able to assist efficient browsing of the consultation/treatment information by presenting the summaries to the medical provider.

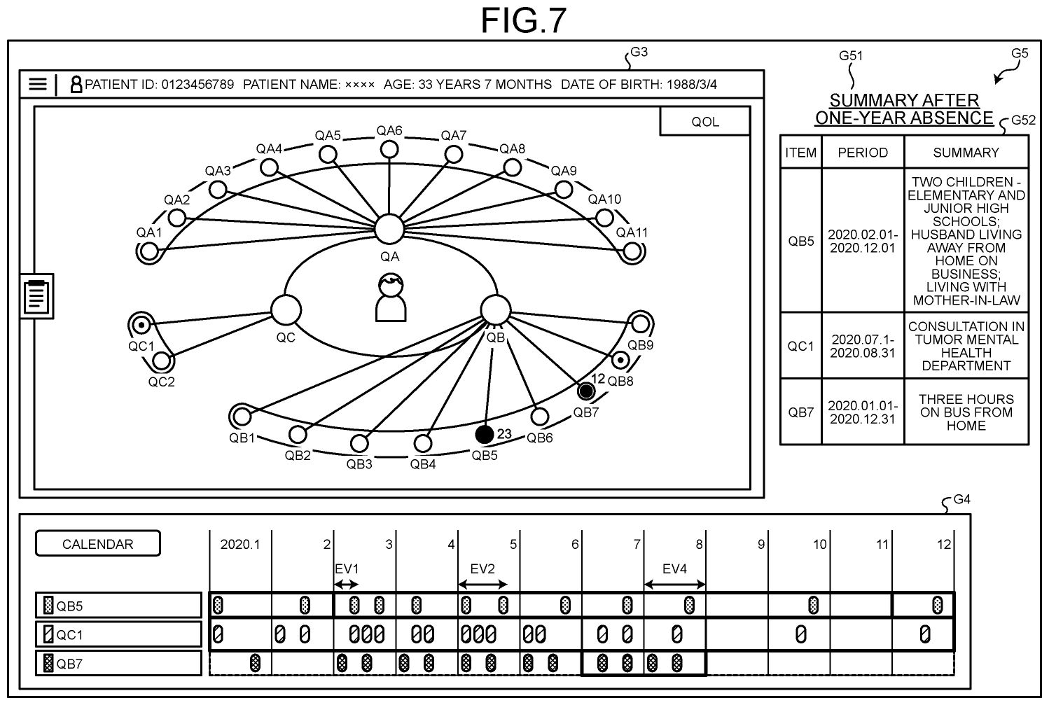

Next, a display example will be explained with reference to FIG. 7, by using a situation where the summary function 757 has generated the summaries according to the specified result obtained by the specifying function 756. FIG. 7 is a drawing illustrating an exemplary screen configuration of a display screen displaying, on a single screen, the second display screen G3, the specified result display screen G4, and the summary display screen G5. As a premise of the following explanation, it is assumed that “Understanding an overall state of the patient” has been input as a reference purpose, and the specifying function 756 has specified “A hospital visit after a one-year absence” as a medical event that had already been implemented (had occurred), on the basis of the specification information 741.

FIG. 7 illustrates the exemplary screen configuration in which the single screen displays the second display screen G3, the specified result display screen G4, and the summary display screen G5. Because the screen configurations of the second display screen G3 and the specified result display screen G4 are the same as those in FIGS. 5 and 6 or the like, explanations thereof will be omitted.

Further, from the second display screen G3 in FIG. 7, illustration of the small category score G33 and the first guides G34 is omitted. Further, on the second display screen G3 in FIG. 7, the item scores G36 and the second guides G37 are displayed only with respect to the items specified by the specifying function 756, namely, the “item QB5”, the “item QB7”, and the “item QC1”. Alternatively, similarly to FIG. 5, the second display screen G3 may display the small category scores G33, the first guides G34, the item scores G36, and the second guides G37.

The specified result display screen G4 in FIG. 7 indicates that the items relevant to “A hospital visit after a one-year absence” specified by the specifying function 756 by referring to the specification information 741 are the “item QB5”, the “item QC1”, and the “item QB7”.

The specified result display screen G4 in FIG. 7 indicates that the reference period of the small events related to the “item QB5” relevant to “A hospital visit after a one-year absence” corresponding to “Understanding an overall state of the patient” is “ten months from February to November”. Further, the specified result display screen G4 in FIG. 7 indicates that the degree of relevance/importance of the “item QB5” relevant to “A hospital visit after a one-year absence” is “high”.

The specified result display screen G4 in FIG. 7 indicates that the reference period of the small events related to the “item QC1” relevant to “A hospital visit after a one-year absence” is “one year from January to December”. Further, the specified result display screen G4 in FIG. 7 indicates that the degree of relevance/importance of the “item QC1” relevant to “A hospital visit after a one-year absence” is “medium”.

The specified result display screen G4 in FIG. 7 indicates that the reference period of the small events related to the “item QB7” relevant to “A hospital visit after a one-year absence” is “two months from July to August”. Further, the specified result display screen G4 in FIG. 7 indicates that the degree of relevance/importance of the “item QB7” corresponding to “A hospital visit after a one-year absence” and “Understanding an overall state of the patient” is “low”.

The summary display screen G5 is a screen for displaying the summaries generated according to the specified result obtained by the specifying function 756. In the example in FIG. 7, the summary display screen G5 includes, as a screen configuration thereof, a summary title G51 and a summary display region G52.

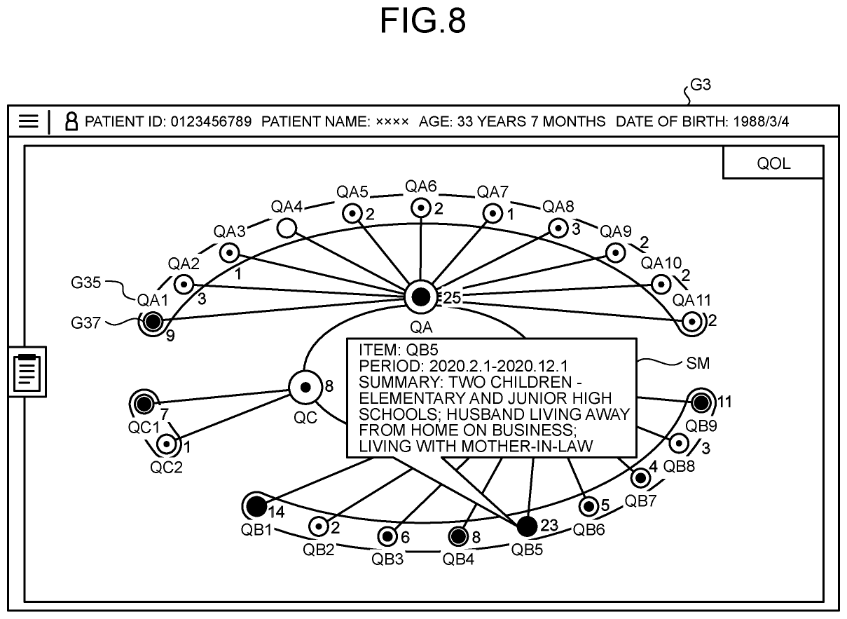

The summary title G51 is a region for displaying a title of the summaries. Displayed in the summary title G51 is the title of the summaries corresponding to the input medical event. In the example in FIG. 8, a title “SUMMARY AFTER ONE-YEAR ABSENCE” corresponding to the specified medical event “A hospital visit after a one-year absence” is displayed.

The summary display region G52 is a region for displaying the summaries generated by the summary function 757 according to the specified result obtained by the specifying function 756. In the example in FIG. 7, an item, a reference period, and a summary are displayed with respect to each of the specified items. Further, in the example in FIG. 7, the specified items such as the “item QB5”, the “item QC1”, and the “item QB7” are displayed in the summary display region G52 in descending order of the specified degrees of relevance/importance.

When the quantity of the specified relevant items exceeds a threshold value (e.g., 3), the summary display region G52 may display only a certain number of (e.g., three) items in descending order of the specified degrees of relevance/importance. Further, when there are two or more items having an equal degree of relevance/importance, the items to be displayed in the summary display region G52 may be determined in accordance with the importance scores thereof.

Further, maximum numbers of characters in the summaries may be varied depending on the degrees of relevance/importance. In that situation, the summary function 757 may be configured to generate the summaries in such a manner that the higher the degree of relevance/importance of an item is, the larger is the maximum number of characters.

Further, on the screen of the specified result display screen G4, the receiving function 753 may be configured to receive, from the medical provider, a correction input to correct the specified result specified by the specifying function 756. Examples of the correction of the specified result include adding an item, deleting an item, correcting the reference periods, and correcting the degrees of relevance/importance. In that situation, the summary function 757 is configured to generate the summaries according to the received correction input.

For example, when the medical provider enters a correction input regarding a reference period on the screen of the specified result display screen G4, the medical provider may enter the correction input to shorten the reference period, by reducing the width of the display frame WE while using an input mechanism such as a mouse. Similarly, the medical provider may enter a correction input to prolong the reference period by expanding the width of the display frame WE.

Further, for example, when the medical provider enters a correction input regarding the degrees of relevance/importance on the screen of the specified result display screen G4, the medical provider may change the display of the display frame WE into a display indicating a desired degree of relevance/importance, by clicking the display frame WE with a mouse or the like, so that the correction input to correct the degree of relevance/importance is received.

Further, the summary function 757 may be configured to generate the summaries regardless of the reference periods of the events specified by the specifying function 756. For example, when the receiving function 753 has received an input designating a topic from the medical provider, the summary function 757 may generate a summary with respect to an item relevant to the designated topic. In this situation, the topic may be the item name itself.

In an example, on the second display screen G3 displayed on the display apparatus 80 illustrated in FIG. 5, when the medical provider clicks using a mouse or touches using a touch panel on an item name (or a second guide G37 corresponding to the item name) displayed in the item display region G35, the receiving function 753 is configured to receive the input designating the item corresponding to the item name. The summary function 757 is configured to generate a summary corresponding to the designated item.

In relation to the above, FIG. 8 is a drawing illustrating an exemplary screen configuration of the second display screen G3 displaying a summary. FIG. 8 illustrates an example of the second display screen G3 in a situation where an input designating the “item QB5” was received from the medical provider. In the example in FIG. 8, a summary SM is displayed in a pop-up window in the vicinity of the item name “item QB5”.

In the example in FIG. 8, the summary SM displays the item name, a time period, and the summary.

The item name indicates the item corresponding to the summary displayed in the summary SM. The time period indicates a reference period of the small events related to the item name displayed in the summary SM. The time period may be set in advance in accordance with the item or may be designated by the medical provider. The summary indicates summarized main points in the contents of the small events related to the item name during the time period displayed in the summary SM.

Although FIG. 8 illustrates the example in which the input designating the single item is received from the medical provider so as to display the single summary SM, it is also acceptable to receive an input designating a plurality of items from the medical provider. In that situation, a plurality of summaries SM will be displayed on the second display screen G3.

Further, the summary SM may automatically be displayed on the second display screen G3 without the medical provider designating an item. For example, the second display screen G3 may automatically display summaries SM corresponding to an arbitrary number of (e.g., three) items in descending order of the importance scores. Further, for example, when there is an item of which the importance score exceeds a threshold value, a summary SM corresponding to such an item may automatically be displayed on the second display screen G3.

The explanation will be continued, returning to the description of FIG. 2. The registering function 758 is configured to perform a process of registering the specification information 741.

For example, via a registration screen for the specification information 741, the receiving function 753 is configured to receive, from the medical provider, inputs of a reference purpose, medical events, items, reference periods, and degrees of relevance/importance. The registering function 758 is configured to store, into the specification information 741 in the memory 740, the reference purpose, the medical events, the items, the reference periods, and the degrees of relevance/importance received by the receiving function 753 so as to be kept in correspondence with one another.

As explained above, because the medical provider is able to freely store, into the specification information 741, the reference purpose, the medical events, the items, the reference periods, and the degrees of relevance/importance so as to be kept in correspondence with one another, it is possible to construct the specification information 741 reflecting a personal idea or the like of each medical provider.

As a result, for example, the medical provider is able to cause a summary to be generated by using, as a relevant item, a certain item that is generally regarded as being irrelevant to a specific medical event, but is personally regarded as being relevant. Further, for example, when a targeted period for summarizing an item relevant to a specific medical event is generally supposed to be one week, but the medical provider personally believes that the targeted period should be two weeks, the medical provider is able to have a summary generated while the targeted period is set to two weeks.

In an example, the registering function 758 may be configured to perform the process of registering (or correcting) the specification information 741 by using a publicly-known machine learning technique or a trained model trained by using a deep learning technique.

In that situation, for example, the registering function 758 is configured to input the reference purpose to the trained model that is functioned, upon receipt of an input of a reference purpose, to output medical events, items, reference periods, and degrees of relevance/importance. The registering function 758 is configured to store the medical events, the items, the reference periods, and the degrees of relevance/importance obtained as a result of inputting the reference purpose to the trained model, into the specification information 741, so as to be kept in correspondence with the input reference purpose.

The trained model described above is trained, for example, by accumulating information related to inputs of reference purposes, medical events, items, reference periods, and degrees of relevance/importance, so as to use the reference purposes as input-side training data and to use the medical events and the like input together with the reference purposes as output-side training data.

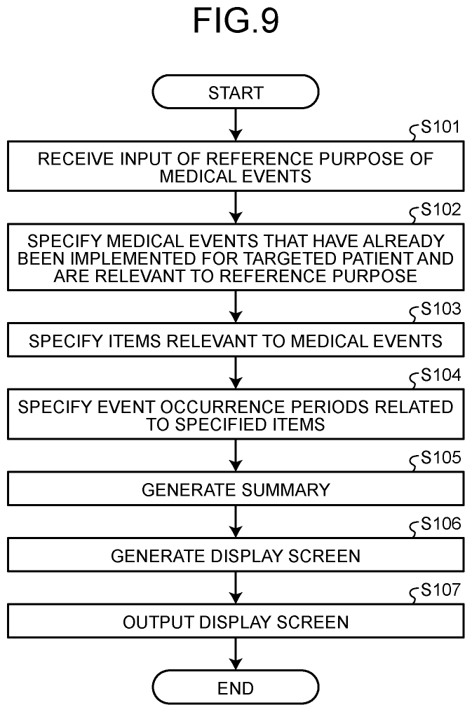

Next, a process performed by the clinical assistance system 1 according to the present embodiment will be explained. FIG. 9 is a flowchart illustrating an example of the process performed by the clinical assistance apparatus 70. As a premise of the explanation of FIG. 9, it is assumed that the display apparatus 80 is displaying the reference purpose input screen, according to an instruction from the medical provider.

To begin with, the receiving function 753 receives an input of a reference purpose from the medical provider (step S101). For example, when the medical provider has entered a selection input to select one of the reference purposes displayed in a reference purpose list by using the input interface 720 such as a mouse or a touch panel, the receiving function 753 receives the contents of the selection input as the input of the reference purpose.

Subsequently, the specifying function 756 specifies medical events that have already been implemented for the targeted patient and are relevant to the reference purpose (step S102). For example, the specifying function 756 specifies the medical events relevant to the reference purpose by referring to the specification information 741. Further, by comparing the medical events relevant to the reference purpose, with the medical events that have already been implemented for the targeted patient and are specified from the consultation/treatment information of the targeted patient, the specifying function 756 specifies the medical events that have already been implemented for the targeted patient and are relevant to the reference purpose.

Subsequently, the specifying function 756 specifies items relevant to the specified medical events (step S103). For example, by referring to the specification information 741 in the memory 740, the specifying function 756 specifies the items corresponding to the medical events specified at step S102 as the items relevant to the medical events.

Subsequently, the specifying function 756 specifies the reference periods of the small events related to the items relevant to the medical events (step S104). For example, by referring to the specification information 741, the specifying function 756 specifies time periods corresponding to the items relevant to the medical events specified at step S103, as the reference periods of the small events related to the items relevant to the medical events.

After that, the summary function 757 generates the summaries (step S105). For example, the summary function 757 generates the summaries on the basis of the contents of the small events that are related to the items specified at step S103 and that occurred during the reference period of the small events specified at step S104.

Subsequently, the generating function 751 generates a display screen (step S106). For example, the generating function 751 generates the summary display screen for displaying the summaries generated at step S105.

After that, the communication controlling function 752 outputs the display screen (step S107), and the present process thus ends. For example, the communication controlling function 752 transmits the summary display screen generated at step S106 to the display apparatus 80. After that, the display apparatus 80 displays the summary display screen.

As explained above, the clinical assistance apparatus 70 according to the present embodiment is configured to receive the input of the reference purpose; configured to specify the reference periods of the small events corresponding to the reference purpose, on the basis of the reference purpose, the specification information 741 keeping reference purposes in correspondence with the reference periods suitable for the reference purposes, and information having chronologically arranged the events related to the items that are classified from the medical biological viewpoint and to the items classified from the other viewpoints; and configured to cause the display apparatus 80 to display the specified result.

With this configuration, for example, the clinical assistance apparatus 70 according to the present embodiment is able to specify the reference period appropriate for browsing the information, with respect to each of the items relevant to the medical events corresponding to the reference purpose input by the medical provider. Consequently, the medical provider is prevented from inadvertently browsing information from a time span having low relevance to the current state of the targeted patient. In other words, the clinical assistance apparatus 70 according to the present embodiment is able to assist the medical provider in efficiently browsing the consultation/treatment information.

Further, the clinical assistance apparatus 70 according to the present embodiment is configured to specify the items relevant to the received reference purpose.

With this configuration, for example, the clinical assistance apparatus 70 according to the present embodiment is able to specify the items relevant to the medical events corresponding to the reference purpose. Consequently, the medical provider is prevented from inadvertently browsing information about items having low relevance to the current state of the targeted patient.

In relation to the above, even when information is browsed about the same medical event, if the purpose of the browsing is different, the appropriate reference period may also be different in some situations. Because the clinical assistance apparatus 70 according to the present embodiment is configured to specify the medical events, the items, and the reference periods in accordance with the reference purpose, it is possible, even in those situations, to specify a reference period appropriate for browsing the information.

Further, the clinical assistance apparatus 70 according to the present embodiment is configured to specify the reference periods of the small events in accordance with the degrees of relevance/importance set with the items.

With this configuration, for example, it is possible to shorten the time period of which the information is to be browsed, with respect to an item having a low degree of relevance/importance with a medical event corresponding to the reference purpose. In other words, the clinical assistance apparatus 70 according to the present embodiment is able to specify the reference period that is more suitable for the medical event corresponding to the reference purpose.

Further, in accordance with the degrees of relevance/importance set with the items, the clinical assistance apparatus 70 according to the present embodiment is configured to exclude certain items from what is to be displayed, even if those items are relevant to a medical event corresponding to the received reference purpose.

With this configuration, for example, even if those items are generally considered to be relevant to the medical event corresponding to the reference purpose, it is possible to exclude the items from what is to be browsed when the degrees of relevance/importance thereof are low. Consequently, the medical provider is prevented from browsing information about the items having low relevance. In other words, the clinical assistance apparatus 70 according to the present embodiment is able to assist the medical provider in efficiently browsing the consultation/treatment information.

Further, it is also possible to carry out the embodiment described above with modifications as appropriate, by changing a part of the configurations or the functions of the apparatuses included in the clinical assistance system 1. Thus, in the following sections, a number of modification examples of the above embodiment will be explained as other embodiments. In the following sections, differences from the above embodiment will primarily be explained, and detailed explanations of some of the features that are the same as those already explained will be omitted. Further, the modification examples described below may be carried out individually or may be carried out in combination, as appropriate.

### First Modification Example

In the embodiment described above, the example was explained in which the items corresponding to the medical events and the reference periods of the small events related to the items are specified and output; however, it is also acceptable to specify and output the reference periods of the small events relevant to the medical events, in units of the small categories.

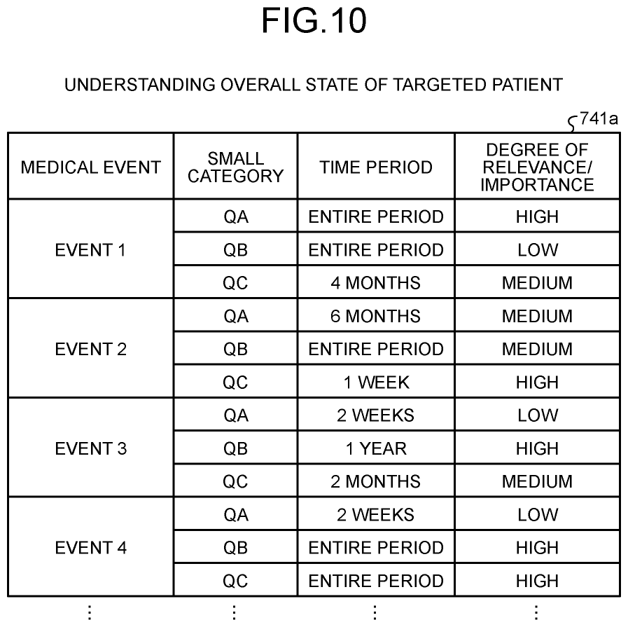

FIG. 10 is a drawing illustrating an exemplary data configuration of specification information according to a first modification example. Specification information 741a in the present modification example is information in which, with respect to each reference purpose, medical events, small categories, reference periods, and degrees of relevance/importance are kept in correspondence with one another.

The small categories denote the small categories of the items illustrated in FIGS. 4 and 5 or the like explained above.

As for the reference periods in the present modification example, reference periods optimal for the small events related to the items are indicated in units of the small categories.

Further, in the present modification example, the degrees of relevance/importance indicate degrees of importance of the items of the medical events relevant to the reference purpose and degrees of relevance between the medical events and the items, in units of the small categories.

FIG. 10 illustrates a correspondence relationship among the medical events, the items, the reference periods, and the degrees of relevance/importance, with respect to the reference purpose “Understanding an overall state of the targeted patient”. Although the example in FIG. 10 indicates only the correspondence relationship with respect to the single reference purpose, it is assumed that such a correspondence relationship is defined with respect to each of different reference purposes.

The first line of FIG. 10 indicates that the reference period of the small events in the item classified in the small category called “small category QA” relevant to “event 1” being a medical event corresponding to the reference purpose “Understanding an overall state of the targeted patient” is “the entire period”. Similarly, it is indicated that the degree of relevance/importance of the items classified in the small category called “QA” is “high”.

In the present modification example, the specifying function 756 is configured to specify the medical events, the small categories, the reference periods, and the degrees of relevance/importance corresponding to the reference purpose received by the receiving function 753, by referring to the specification information 741a.

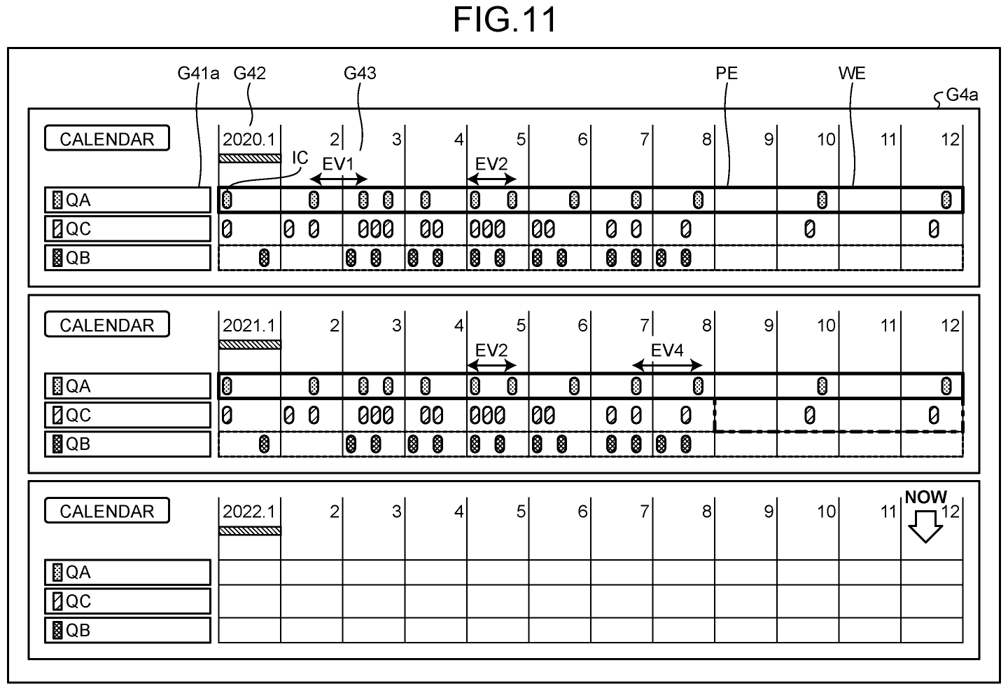

In relation to the above, FIG. 11 illustrates an exemplary screen configuration of a specified result display screen G4a according to the first modification example.

In the example in FIG. 11, the specified result display screen G4a displays a specified result including items and reference periods of the small events from three years obtained by the specifying function 756, in separate sections each corresponding to one year. The specified result display screen G4a includes, as a screen configuration thereof, a small category display region G41a, the calendar display region G42, the event display region G43, the event icons IC, and the display frame WE. Because the calendar display region G42, the event display region G43, the event icons IC, and the display frame WE are the same as those in FIG. 6 explained above, explanations thereof will be omitted.

The small category display region G41a is a region for displaying the small categories specified by the specifying function 756. In the example in FIG. 11, three small category names such as “small category QA”, “small category QB”, and “small category QC” are displayed.

As an example, a situation will be explained in which “Understanding an overall state of the targeted patient” has been input as a reference purpose. In this situation, FIG. 11 indicates that the reference period of the small events related to the items classified in the “small category QA” relevant to “event 1” corresponding to “Understanding an overall state of the targeted patient” is “two years from January 2020 to December 2021”.

In this situation, in the example in FIG. 11, there is no consultation/treatment information of the targeted patient for January 2022 to the present time. In that situation, a base point of the reference period of the small events may be set to the time at which a small event was implemented (or occurred) most recently, instead of the present time.

Further, FIG. 11 indicates that the degree of relevance/importance of the items classified in the “small category QA” relevant to “event 1” is “high”.

In the present modification example, the summary function 757 is configured to generate summaries in units of the small categories according to the specified result obtained by the specifying function 756. Alternatively, the summary function 757 may be configured to generate summaries in units of the items, similarly to the embodiment described above.

According to the present modification example, when a medical provider wishes to browse the consultation/treatment information in a wide and shallow manner, for instance, it is possible to assist efficient browsing of the consultation/treatment information.

### Second Modification Example

In the above embodiment and the first modification example, the example was explained in which the specification information is defined by using the table structure. However, the specification information may be defined by using a graph structure.

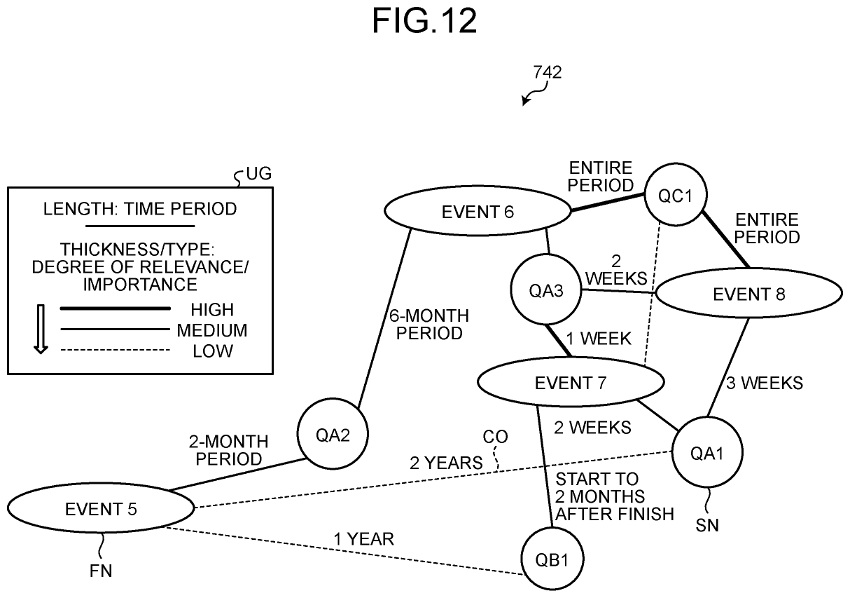

FIG. 12 illustrates an example of the graph structure of specification information 742 according to a second modification example. In the example in FIG. 12, the specification information 742 is structured with a plurality of nodes and information indicating connections between the nodes with magnitudes of the degrees of relevance/importance and relevant periods. In this situation, the graph structure of the specification information 742 may be defined for each of different reference purposes.

For example, the plurality of nodes may be classified into first nodes FN representing medical events and second nodes SN representing items classified from the medical biological viewpoint, items classified from the psychological viewpoint, items classified from the social viewpoint, and the like.

In the example in FIG. 12, events 5 to 8 are the first nodes FN, whereas the items QA1 to QA3, the item QB1, and the item QC1 are the second nodes SN. Further, the first nodes FN and the second nodes SN are connected by using straight lines CO expressing the connection between the nodes.

Further, a legend UG is information indicating relationships among the nodes defined by the straight lines CO. The legend UG is presented for the sake of convenience in the explanation and is not included in the specification information 742.

In the example in FIG. 12, the legend UG indicates that the thicknesses of the lines express the degrees of relevance/importance between the medical events and the items. In FIG. 12, the thicknesses of the lines are in three different levels, and the thinnest line is drawn as a dotted line.

Further, in the example in FIG. 12, the legend UG indicates that the lengths of the lines express the reference periods of the items corresponding to the medical events. In FIG. 12, it should be noted, however, that when the length of a time period is variable, such as when the reference period is “the entire period”, information indicating the situation is appended to a line having a predetermined length.

As an example, in FIG. 12, the degree of relevance/importance between event 5 and the item QA1 is “low”, while the reference period of the item QA1 for event 5 is “two years”.

In the following sections, by using an example in which the graph structure is defined for each of different reference purposes, a process of specifying items of medical events, reference periods of the items, and degrees of relevance/importance by using the specification information 742 will be explained.

In this situation, to begin with, the specifying function 756 is configured to specify, from the specification information 742, a graph structure corresponding to the reference purpose input by a user. Subsequently, the specifying function 756 is configured to refer to the consultation/treatment information managed in a format having information arranged in a chronological order and to further extract certain first nodes corresponding to the medical events implemented for a targeted examined subject, from among the first nodes FN in the specified graph structure.

Further, by specifying certain second nodes SN connected by straight lines CO to the extracted first nodes FN, the specifying function 756 is configured to specify the items of the extracted medical events. After that, on the basis of the thicknesses and the lengths of the straight lines CO in the graph, the specifying function 756 is configured to specify reference periods and degrees of relevance/importance of the items.

Alternatively, the specification information 742 may have the nodes arranged in such a manner that the distances between the nodes in the graph express the strengths of the relationships among the nodes. With this configuration, it is possible to define not only the relationships between the first nodes FN and the second nodes SN, but also the relationships of the first nodes FN to each other and the relationships of the second nodes SN to each other.

With these configurations, for example, it is possible to extract only certain medical events having high relevance, from among the plurality of medical events implemented for the targeted examined subject and to extract certain items that are not the items of a specific medical event but are highly relevant to those items.

In the present modification example, for instance, the relationships between the medical events and the items classified from the medical biological viewpoint, the items classified from the psychological viewpoint, the items classified from the social viewpoint, and the like are defined by using the lengths and the thicknesses of the lines. Consequently, according to the present modification example, it is possible to define the degrees of relevance/importance and the reference periods in a detailed manner, by simply adjusting the thicknesses and the lengths of the lines.

Further, as for parameters expressing the reference periods of the items corresponding to the lengths of the lines in the graph and the degrees of relevance/importance corresponding to the thicknesses of the lines may be directly changed as a result of the user changing the numerical values of the parameters or may be indirectly set or changed as a result of editing the thicknesses and/or the lengths of the lines expressed in the graph. In another example, the parameters may be set by the system, by using a method by which a plurality of parameter sets each indicating a reference period and a degree of relevance/importance are defined in correspondence with attributes of examined subjects or with different users who use the present system, so as to establish a setting in a collective manner by reading one of the defined parameter sets.

Further, for example, because the relationships among the nodes are defined by using the arranged positions of the nodes, it is possible to more easily perform the process of extracting relevant items, or the like, as compared to the situation where the relationships between the medical events and the items are defined by using the table structure.

### Third Modification Example

In the above embodiment, the example was explained in which the medical events and the reference purpose that are manually input by the medical provider are received; however, another configuration is also acceptable in which medical events and a reference purpose are automatically set on the basis of a conversation between a medical provider and the targeted patient or the like.

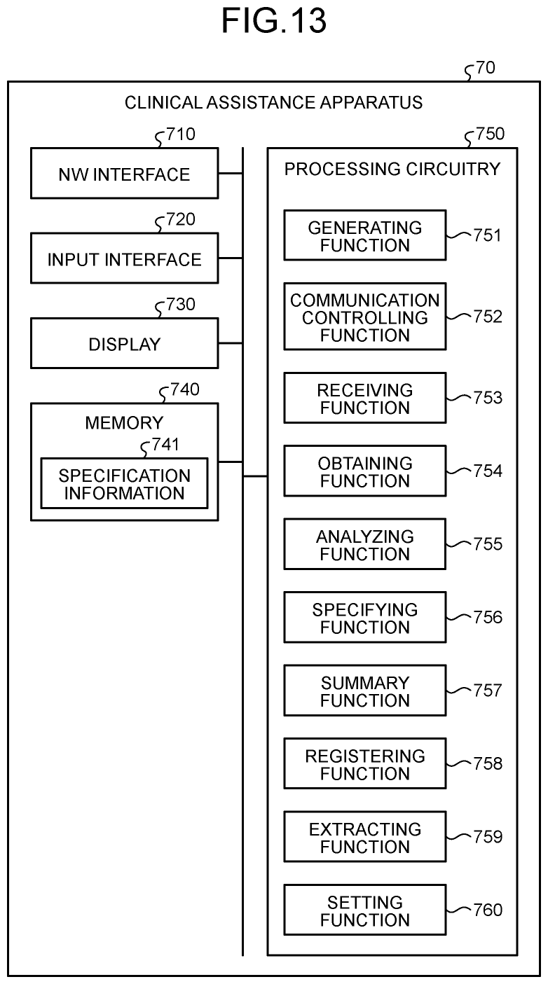

FIG. 13 is a block diagram illustrating an exemplary configuration of the clinical assistance apparatus 70 according to a third modification example. The clinical assistance apparatus 70 according to the present modification example includes the NW interface 710, the input interface 720, the display 730, the memory 740, and the processing circuitry 750. Because the NW interface 710, the input interface 720, the display 730, and the memory 740 are the same as those in the above embodiment, explanations thereof will be omitted.

For example, the processing circuitry 750 according to the present modification example includes, as functional units thereof, the generating function 751, the communication controlling function 752, the receiving function 753, the obtaining function 754, the analyzing function 755, the specifying function 756, the summary function 757, the registering function 758, an extracting function 759, and a setting function 760. Because the generating function 751, the communication controlling function 752, the receiving function 753, the obtaining function 754, the analyzing function 755, the specifying function 756, the summary function 757, and the registering function 758 are substantially the same as those in the above embodiment, explanations thereof will be omitted.

The extracting function 759 is configured to extract keywords relevant to a topic, from information related to a conversation between the medical provider and the targeted patient.

For example, when the receiving function 753 has received an instruction to extract the keywords from the medical provider, the extracting function 759 is configured to extract the keywords relevant to the reference purpose from the conversation information 51 obtained by the obtaining function 754, by using a publicly-known natural language processing technique.

In this situation, the extracted keywords may include not only those related to a life prognosis (how long he/she can live) of the targeted patient, but also those related to a functional prognosis. Examples of the functional prognosis include a motion prognosis (how long he/she is able to move freely), a cognition prognosis (how long he/she is able to have complex thoughts), an eating prognosis (how long he/she is able to enjoy eating), and a conversation prognosis (how long he/she is able to converse with others comfortably).

In an example, together with the abovementioned instruction to perform the extraction, the receiving function 753 may also receive, from the medical provider, a designation of a time period from which the keywords are to be extracted.

The setting function 760 is configured to set the reference purpose on the basis of the information related to the conversation between the medical provider and the targeted patient. The setting function 760 is an example of a setting unit. For example, the setting function 760 is configured to set the reference purpose on the basis of the keywords extracted by the extracting function 759.

In an example, by using medical ontology or the like, the setting function 760 is configured to search for synonyms, equivalent terms, and the like, of the extracted keywords, while using the extracted keywords as search words. After that, on the basis of a result of the search and a list of reference purposes that is set in advance, the setting function 760 is configured to automatically set the reference purpose.

With this configuration, the setting function 760 is able to set the reference purpose in accordance with how the targeted patient wishes to lead a life from now on.

The specifying function 756 according to the present modification example is configured to specify items, reference periods, and degrees of relevance/importance corresponding to the reference purpose set automatically by the setting function 760. In an example, when the reference purpose has automatically been set, the generating function 751 may be configured to generate a display screen different from that generated when a manually-input reference purpose is received.

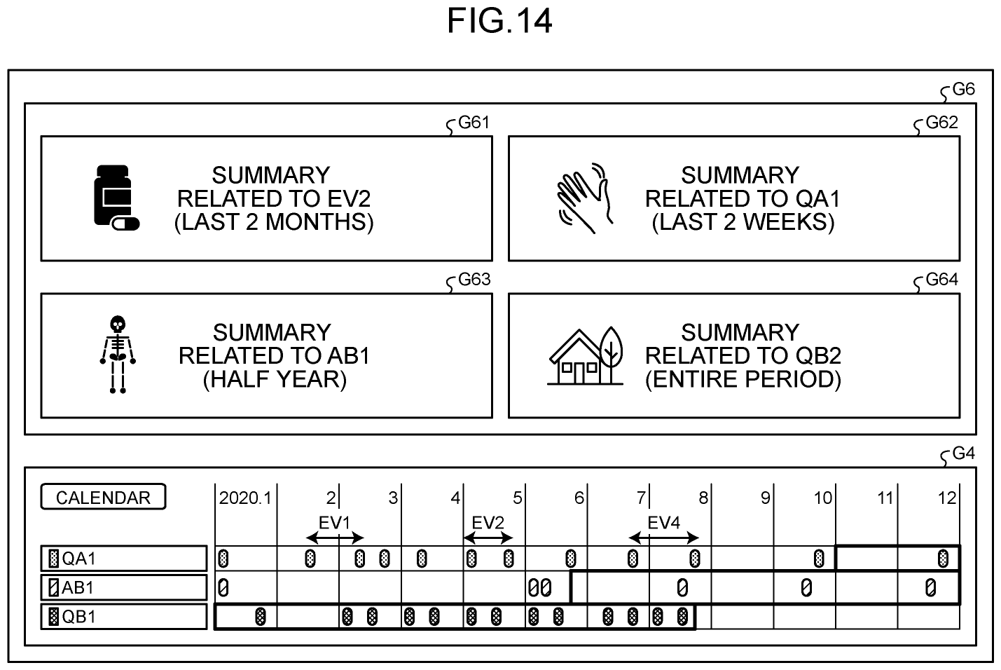

In relation to the above, FIG. 14 is a drawing illustrating an exemplary screen configuration of a display screen used when a reference purpose is automatically set. The display screen in FIG. 14 includes, as a screen configuration thereof, the specified result display screen G4 and summary display buttons G6. Because the specified result display screen G4 is the same as that in FIG. 6 and the like, explanations thereof will be omitted.

The summary display buttons G6 are buttons for causing summaries to be displayed that are relevant to the keywords extracted by the extracting function 759. Displayed as the summary display buttons G6 are buttons for displaying a summary about the medical events specified by the specifying function 756 on the basis of the reference purpose set automatically by the setting function 760, a summary about a certain item having a high degree of relevance/importance among the items specified by the specifying function 756, and the like. Further, each of the buttons displays a title of the summary and a targeted period of the summary.

Further, as one of the summary display buttons G6, it is also acceptable to display a button for causing a summary to be displayed about a certain item that is highly relevant to the keywords extracted by the extracting function 759.

In the example in FIG. 14, displayed as the summary display buttons G6 are a button G61 for displaying a summary relevant to event 1, a button G62 for displaying a summary relevant to the item QA1, a button G63 for displaying a summary relevant to an item AB1, and a button G64 for displaying a summary relevant to the item QB2.

The button G61 displays a summary title “Summary relevant to event 1” and a targeted period of the summary which is the “last two months”. The button G62 displays a summary title “Summary relevant to QA1” and a targeted period of the summary which is the “last two weeks”. The button G63 displays a summary title “Summary relevant to AB1” and a targeted period of the summary which is “half a year”. The button G64 displays a summary title “Summary relevant to QA2” and a targeted period of the summary which is the “the entire period”.

For example, when one of the buttons is pressed by the medical provider, the display apparatus 80 displays a display screen for displaying the summary corresponding to the pressed button. For example, when the button G61 is pressed, the display apparatus 80 displays the same display screen as in FIG. 8.

According to the present modification example, without the medical provider having to manually input the reference purpose, it is possible to specify the items relevant to the keywords extracted from the conversation with the targeted patient and the reference periods of the small events related to the items. Consequently, the medical provider is able to browse consultation/treatment information that is highly relevant to the extracted keywords. In other words, according to the present modification example, it is possible to assist the medical provider in efficiently browsing the consultation/treatment information.

### Fourth Modification Example

In the above embodiment and modification examples, the examples were explained in which the medical provider browses the consultation/treatment information; however, it is also acceptable to enable not only the medical provider but also the targeted patient to browse the consultation/treatment information.

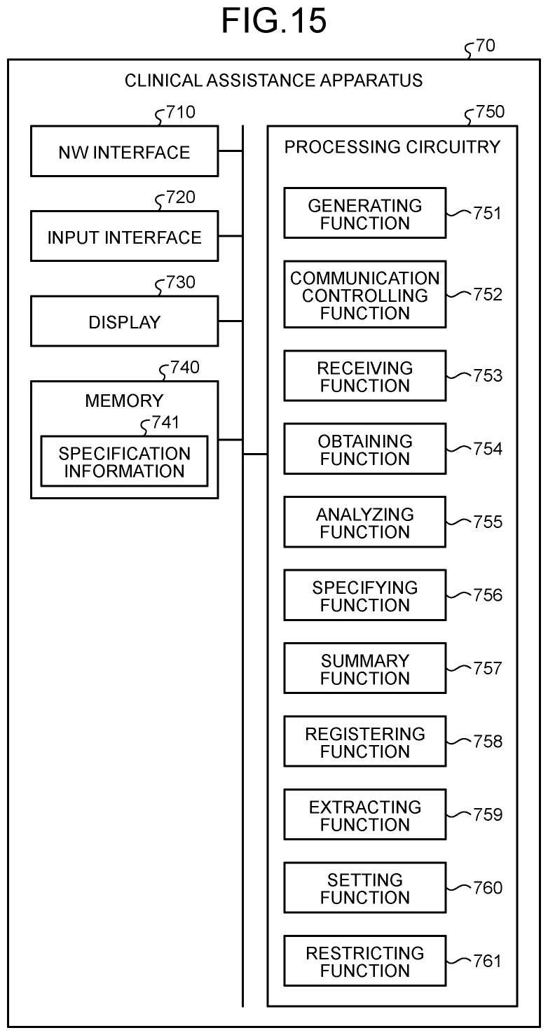

FIG. 15 is a block diagram illustrating an exemplary configuration of the clinical assistance apparatus 70 according to a fourth modification example. The clinical assistance apparatus 70 according to the present modification example includes the NW interface 710, the input interface 720, the display 730, the memory 740, and the processing circuitry 750. Because the NW interface 710, the input interface 720, the display 730, and the memory 740 are the same as those in the third modification example described above, explanations thereof will be omitted.

For example, the processing circuitry 750 according to the present modification example includes, as functional units thereof, the generating function 751, the communication controlling function 752, the receiving function 753, the obtaining function 754, the analyzing function 755, the specifying function 756, the summary function 757, the registering function 758, the extracting function 759, the setting function 760, and a restricting function 761. Because the generating function 751, the communication controlling function 752, the receiving function 753, the obtaining function 754, the analyzing function 755, the specifying function 756, the summary function 757, the registering function 758, the extracting function 759, and the setting function 760 are substantially the same as those in the third modification example described above, explanations thereof will be omitted.

The restricting function 761 is configured to set browsing authority of the consultation/treatment information. For example, the restricting function 761 is configured to set, with respect to each of the items, whether the targeted patient is able or unable to browse. In an example, the restricting function 761 may be configured to set, with respect to each of the items, whether the browsing is permitted or not, in accordance with attributes of medical providers (medical doctors, nurses, radiology technologists, clinical technologists, etc.).

In the present modification example, the receiving function 753 is configured to receive, from the medical provider, an instruction to switch between browsing modes. Examples of the browsing modes include a medical provider mode in which only medical providers browse the consultation/treatment information and a patient mode in which medical providers and the targeted patient browse the consultation/treatment information. In an example, when the browsing mode is set to the patient mode, the restricting function 761 is configured to restrict outputs of information related to certain items having a setting that prohibits browsing of the targeted patient.

Further, when the browsing mode is set to the patient mode, the generating function 751 according to the present modification example may be configured to generate a display to present the targeted patient with activity levels indicating the extent of activities which the targeted patient is able to perform, such as a travel level, a motion level, a cognition level, an eating level, and a conversation level of the targeted patient.

The travel level denotes, for example, evaluating up to which level the targeted patient is able to travel, in accordance with indices expressing degrees of difficulty in traveling.

The motion level denotes, for example, evaluating up to which level the targeted patient is able to make motion, in accordance with indices expressing degrees of difficulty in motion.

The cognition level denotes, for example, evaluating up to which level the targeted patient is able to have cognition, in accordance with indices expressing degrees of difficulty in cognition.

The eating level denotes, for example, evaluating up to which level the targeted patient is able to eat, in accordance with indices expressing degrees of difficulty in eating.

The conversation level denotes, for example, evaluating up to which level the targeted patient is able to have conversations, in accordance with indices expressing degrees of difficulty in conversing.

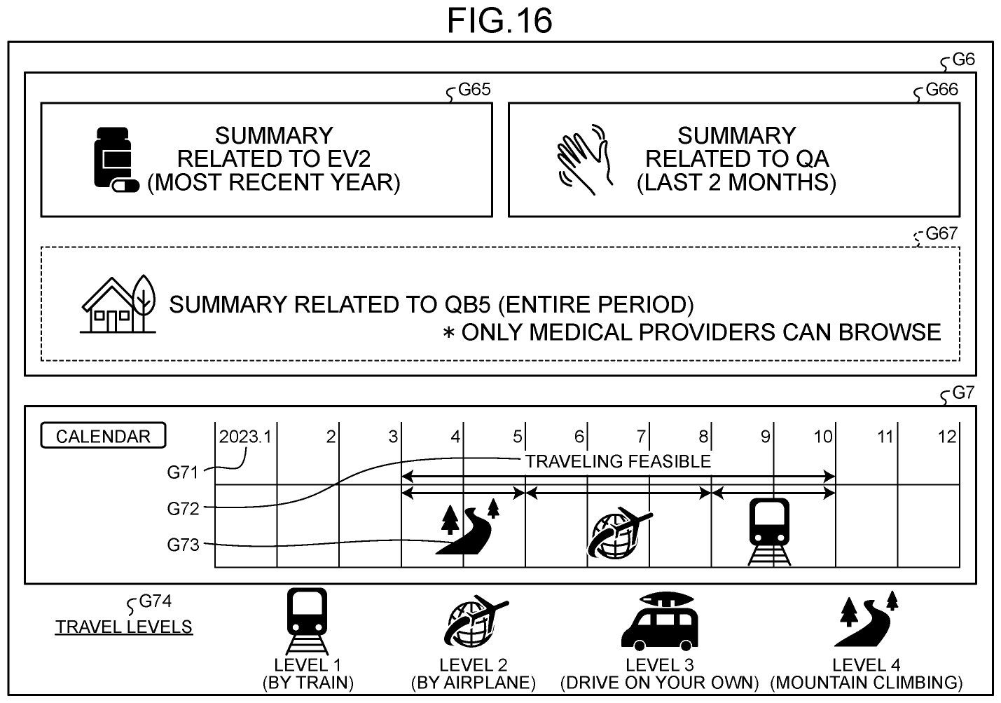

In relation to the above, FIG. 16 is a drawing illustrating an exemplary screen configuration of a display screen in the situation where only information browsable by the targeted patient is displayed. The display screen in FIG. 16 includes, as a screen configuration thereof, summary display buttons G6 and an activity level display screen G7.

In the example in FIG. 16, displayed as the summary display buttons G6 are: a button G65 for displaying a summary relevant to event 2; a button G66 for displaying a summary relevant to QA; and a button G67 for displaying a summary relevant to QB5. Among these, because the summary relevant to QB5 contains information which the targeted patient is not permitted to browse, the button G67 is displayed in a mode that does not permit pressing.

The button G65 displays a summary title “Summary relevant to event 2” and a targeted period of the summary which is “the most recent year”. The button G66 displays a summary title “Summary relevant to QA” and a targeted period of the summary which is “the last two weeks”. The button G67 displays a summary title “Summary relevant to QB5” and a targeted period of the summary which is “the entire period”.

The activity level display screen G7 is a screen for displaying the activity levels of the targeted patient. The activity level display screen G7 in FIG. 16 includes, as a screen configuration thereof, a calendar display region G71, a feasible time display region G72, an activity level display region G73, and an explanation display region G74.

The calendar display region G71 is a region for displaying a calendar that presents the targeted patient with time spans in which activities are feasible. The calendar display region G71 displays a calendar later than the present time. In the example in FIG. 16, the calendar for January to December, 2023 is displayed.

The feasible time display region G72 is a region for presenting the targeted patient with the time spans when the activities relevant to the keywords extracted by the extracting function 759 are feasible. In the example in FIG. 16, the feasible time display region G72 indicates that traveling is feasible in March to July, 2023.

The activity level display region G73 is a region for displaying the activity levels of the targeted patient. In the example in FIG. 16, a travel level of the targeted patient in March to July, 2023 is displayed with an icon, for each time span.

The activity level display region G73 in FIG. 16 indicates that traveling on level 4 is feasible in March to April, 2023. Further, the activity level display region G73 indicates that traveling on level 2 is feasible in May to August, 2023. Also, the activity level display region G73 indicates that traveling on level 1 is feasible in September to October, 2023.

The explanation display region G74 is a display region for displaying explanations of the icons indicating the activity levels. The example in FIG. 16 indicates that travel level 1 denotes “travel by train is feasible”; travel level 2 denotes “travel by airplane is feasible”; travel level 3 denotes “travel by driving an automobile on your own is feasible”; and travel level 4 denotes “travel involving a large physical burden such as mountain climbing is also feasible”.

As explained above, by presenting the activities that are feasible for the targeted patient in a visualized manner, the clinical assistance apparatus 70 according to the present modification example is able to assist the targeted patient in activities for understanding his/her own state.

According to the present modification example, it is possible to assist efficiently browsing the consultation/treatment information, not only when the consultation/treatment information is browsed by medical providers, but also when browsed by the targeted patient.

According to at least one aspect of the embodiments and the like described above, it is possible to assist medical providers in efficiently browsing the consultation/treatment information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured:
to receive an input of a topic related to a targeted patient subject to a consultation/treatment process;
to specify an item of a medical event relevant to the input topic and a reference period of the item, from time-series information recording medical events implemented for the targeted patient and time spans in each of which a state of a different one of items was observed from the targeted patient during the medical event, the items belonging to the medical events and being classified from a medical biological viewpoint and a viewpoint other than the medical biological viewpoint; and
to output a specified result.

2. The medical information processing apparatus according to claim 1, wherein the processing circuitry is configured to specify the item of the medical event relevant to the input topic and the reference period of the item, from time-series information, on a basis of correspondence information which, with respect to each of a plurality of topics, associates the medical event relevant to the topic with the reference period of each of the items belonging to the medical event.

3. The medical information processing apparatus according to claim 1, wherein
the processing circuitry is configured to receive the input of the topic including a reference purpose of consultation/treatment information about the consultation/treatment process for the targeted patient,
the processing circuitry is configured to specify the item of the medical event relevant to the reference purpose and the reference period of the item, on the basis of the time-series information, and
the processing circuitry is configured to output the item of the medical event relevant to the specified reference purpose and the reference period of the item, as the specified result.

4. The medical information processing apparatus according to claim 3, wherein
the processing circuitry is configured to automatically set the reference purpose on a basis of information obtained from the targeted patient, and
the processing circuitry is configured to specify the item of the medical event relevant to the reference purpose and the reference period of the item, on a basis of the set reference purpose.

5. The medical information processing apparatus according to claim 1, wherein
the processing circuitry is configured to specify the item of the medical event set with a weight expressing a degree of strength of a relationship with the topic and the reference period of the item, and
the processing circuitry is configured to specify the reference period of the item of the medical event corresponding to the weight.

6. The medical information processing apparatus according to claim 5, wherein, when the weight satisfies a predetermined condition, the processing circuitry is configured to exclude the item of the medical event relevant to the topic from what is to be output.

7. The medical information processing apparatus according to claim 5, wherein
the processing circuitry is configured to specify the item of the medical event and the reference period of the item, the item being classified into a first item classified from a medical biological viewpoint and a second item classified from a viewpoint other than the medical biological viewpoint, and
together with the specified result, the processing circuitry is configured to output a reference period of the first item, a reference period of the second item, a weight on the first item, and a weight on the second item, in a distinguishable manner.

8. The medical information processing apparatus according to claim 7, wherein the processing circuitry is configured to output a first event indicating a state of the first item in each of different time spans during a time period corresponding to the reference period of the specified first item and a second event indicating a state of the second item in each of different time spans during a time period corresponding to the reference period of the specified second item, so as to be arranged in a chronological order.

9. The medical information processing apparatus according to claim 8, wherein
the processing circuitry is configured to receive an input to select an event from among two or more of the first and the second events, and
the processing circuitry is configured to output details of the selected first or second event.

10. The medical information processing apparatus according to claim 1, wherein the processing circuitry is configured to specify a reference period of the item of the medical event corresponding to a most recent consultation date/time of the targeted patient.

11. The medical information processing apparatus according to claim 1, wherein
the processing circuitry is configured to receive an input of the topic, the medical event relevant to the topic, and a reference period of each of items belonging to the medical event, and
the processing circuitry is configured to store, into a memory apparatus, the received topic, the medical event relevant to the topic, and the reference period of each of the items belonging to the medical event so as to be kept in correspondence with one another.

12. A medical information display system comprising a medical information processing apparatus and a medical information display apparatus, wherein
the medical information processing apparatus includes processing circuitry configured:
to receive an input of a topic related to a targeted patient subject to a consultation/treatment process;
to specify an item of a medical event relevant to the input topic and a reference period of the item, from time-series information recording medical events implemented for the targeted patient and time spans in each of which a state of a different one of items was observed from the targeted patient during the medical event, the items belonging to the medical events and being classified from a medical biological viewpoint and a viewpoint other than the medical biological viewpoint; and
to transmit a specified result obtained to the medical information display apparatus, and
the medical information display apparatus includes processing circuitry configured:
to receive the specified result; and
to exercise control so as to have the specified result displayed by a display apparatus.

13. A medical information processing method being implemented by a medical information processing apparatus and comprising:
receiving an input of a topic related to a targeted patient subject to a consultation/treatment process;
specifying an item of a medical event relevant to the input topic and a reference period of the item, from time-series information recording medical events implemented for the targeted patient and time spans in each of which a state of a different one of items was observed from the targeted patient during the medical event, the items belonging to the medical events and being classified from a medical biological viewpoint and a viewpoint other than the medical biological viewpoint; and
outputting a specified result specified at the specifying step.
